(19) United States
(12) Patent Application Publication
(10) Pub. No.: US 2024/0197918 A1
(43) Pub. Date: Jun. 20, 2024

(54) TISSUE-TARGETED MODIFIED AAV CAPSIDS AND METHODS OF USE THEREOF

(71) Applicant: Locanabio, Inc., San Diego, CA (US)

(72) Inventor: Greg NACHTRAB, San Diego, CA (US)

(73) Assignee: Locanabio, Inc., San Diego, CA (US)

(21) Appl. No.: 18/287,963

(22) PCT Filed: Apr. 22, 2022
(86) PCT No.: PCT/US2022/026048

§ 371 Date: Oct. 23, 2023

Related U.S. Application Data
(60) Provisional application No. 63/299,697, filed on Jan. 14, 2022.
(60) Provisional application No. 63/178,965, filed on Apr. 23, 2021.

(51) Int. Cl. A61K 48/00; C07K 14/005; C12N 15/86

(52) U.S. Cl. CPC A61K 48/0058; C07K 14/005; C12N 15/86; C12N 2750/14122; C12N 2750/14143; C12N 2750/14145

(57) ABSTRACT

Provided herein are modified AAV capsids comprising modified AAV capsid proteins having improved transduction in muscle tissue, reduced immunogenicity, reduced neutralizing antibody binding, reduced liver tissue transduction, and combinations thereof. Also provided are methods of treating a patient with AAV viral vectors comprising modified AAV capsid proteins.

### FIELD OF THE DISCLOSURE

The disclosure is directed to molecular biology, gene therapy, and compositions and methods for enhancing transduction of AAV capsids comprising modified AAV capsid proteins. In particular, modified AAV capsids comprising modified AAV capsid proteins are provided having enhanced transduction is muscle tissue and/or reduced transduction in liver tissue.

### CROSS-REFERENCE TO RELATED APPLICATIONS

This application claims priority to, and the benefit of, U.S. Provisional Application No. 63/178,965 filed Apr. 23, 2021 and U.S. Provisional Application No. 63/299,697 filed Jan. 14, 2022. The contents of each of which is hereby incorporated by reference in their entireties.

### INCORPORATION BY REFERENCE OF SEQUENCE LISTING

The contents of the text file named “LOCN_011_001WO_SeqList_ST25”, which was created on Apr. 21, 2022 and is 1.33 MB in size, are hereby incorporated by reference in their entirety.

### BACKGROUND

Adeno-associated virus (AAV) vector-based therapeutics can have limited transduction in specific tissue types and transduction can be limited due to pre-existing neutralizing antibodies (NAb) specific for specific AAV serotypes.

In particular, AAV8 and AAV9 are commonly used vectors for therapies delivered for skeletal muscle transduction via systemic injection. However, these serotypes also transduce the liver and a significant percentage of the patient population has a moderate to high NAb titer against AAV8 or AAV9. As such, there exists a need for the development of AAV capsids having modified AAV capsid protein having enhanced tissue-specific transduction in a desired tissue and/or reduced transduction in a non-desired tissue. In some aspects, it is desired to have enhanced transduction in muscle tissue and/or reduced transduction in liver tissue.

Disclosed herein are AAV capsids comprised of modified and chimeric AAV capsid protein sequences with enhanced muscular, transduction, as well as decreased transduction in liver tissue. In some aspects, AAV capsids comprised of modified and chimeric AAV capsid protein sequences have reduced sensitivity to pre-existing neutralizing antibodies.

### SUMMARY

The disclosure provides a modified AAV capsid protein comprising a modified variable region (VR) VIII. In some aspects, the capsid protein is a VP1, VP2, or VP3 capsid protein. In some aspects, the modified VR VIII comprises a peptide insertion.

In some aspects, the peptide insertion comprises an RGD-motif peptide insertion. In some aspects, the RGD-motif insertion comprises RGDLGLS (SEQ ID NO: 303), RGDLSTP (SEQ ID NO: 304), SNSRGDYNSL (SEQ ID NO: 305), ENRRGDFNNT (SEQ ID NO: 306), SRGDYNSL (SEQ ID NO: 307), RGDYNSL (SEQ ID NO: 308), RGDLST (SEQ ID NO: 309), or RGDYVGL (SEQ ID NO: 310).

In some aspects, the RGD-motif insertion comprises a variable domain of camelid heavy-chain-only antibody (VHH) RGD peptide. In some aspects, the VHH RGD peptide comprises an amino acid sequence at least 85%, 90%, 95%, 96%, 97%, 98%, 99% or 100% identical to the amino acid sequence EVQLQASGGGFVQAGGSLRLSCAVSGRGDLSTPSYGMHWVRQAPGKEREFVAGISR GDYNSLYYADSVQGRFTISRDNAKNTVYLQMNSLKPEDTATYYCAENRRGDFNNT YWGQGTQVTVSS (SEQ ID NO: 316).

In some aspects, the peptide insertion comprises an acetylcholinesterase collagenic tail (ColQ) peptide. In some aspects, the ColQ peptide comprises an amino acid sequence at least 85%, 90%, 95%, 96%, 97%, 98%, 99% or 100% identical to the amino acid sequence TPFYPVGYTVKQPGTCGDGVLQPGEECDDGNPDVSDGCIDCHRAYCGDGYRHQGV EDCDGSDFGYLTCETYLPGSYGDLRCTQYCSIDSTPCRYFT (SEQ ID NO: 302).

In some aspects, the peptide insertion comprises one or more linker sequences at the N-terminus or C-terminus of the inserted peptide. In some aspects, the linker sequence comprises GGGGS (SEQ ID NO: 311), GGGGSGGGGS (SEQ ID NO: 312); GGGGSGGGGSGGGGS(SEQ ID NO: 313); GGGGSGGGGSGGGGSGGGGS (SEQ ID NO: 314); or GGGGSGGGGSGGGGSGGGGSGGGGS (SEQ ID NO: 315).

In some aspects, the AAV serotype is AAV9 or AAVRh74.

In some aspects, the modified AAV capsid protein further comprises one or more amino acid mutations. In some aspects, the one or more amino acid mutations reduce liver transduction. In some aspects, the mutations comprise: i) at least one of F503I, G507I, Y707C and/or Y708C of Rh74, or ii) N498I of AAV9.

In some aspects, the capsid protein comprises an amino acid sequence at least 85%, 90%, 95%, 96%, 97%, 98%, 99% or 100% identical to the amino acid sequence set forth in any one of SEQ ID NOs: 53, 75, 102, 124, 245, 249, 255, 258, 262, or 267.

The disclosure provides a modified AAV capsid protein comprising an amino acid sequence at least 85%, 90%, 95%, 96%, 97%, 98%, 99% or 100% identical to the amino acid sequence set forth in any one of SEQ ID NOs: 53, 75, 102, 124, 245, 249, 255, 258, 262, or 267.

In some aspects, the capsid protein is encoded by a nucleic acid sequence at least 85%, 90%, 95%, 96%, 97%, 98%, 99% or 100% identical to the amino acid sequence set forth in any one of SEQ ID NOs: 151, 173, 199, 221, 270, 274, 280, 283, 287, or 292.

The disclosure provides an AAV capsid comprising one or more AAV capsid proteins according to any embodiment of the disclosure.

In some aspects, the AAV capsid has enhanced transduction in a targeted tissue or cell type relative to other tissue or cell types. In some aspects, the targeted tissue type is muscular tissue or muscle cells. In some aspects, the AAV capsid has enhanced transduction in muscle tissue. In some aspects, the AAV capsid has reduced transduction in non-targeted tissues or cell types. In some aspects, the non-targeted tissues include liver, lung, kidney, brain, spleen, intestine, spinal cord, or reproductive organs.

In some aspects, the transduction in muscle tissue is enhanced by at least about 10%, about 20%, about 30%, about 40%, about 50%, about 100%, about 200%, or about 300% compared the parental and/or unmodified AAV capsid.

In some aspects, the capsid comprises: i) a VP1 capsid protein comprising an amino acid sequence at least 85%, 90%, 95%, 96%, 97%, 98%, 99% or 100% identical to the amino acid sequence set forth in SEQ ID NO: 53; and ii) a VP2/VP3 capsid protein comprising an amino acid sequence at least 85%, 90%, 95%, 96%, 97%, 98%, 99% or 100% identical to the amino acid sequence set forth in SEQ ID NO: 102.

In some aspects, the capsid comprises: i) a VP1 capsid protein comprising an amino acid sequence at least 85%, 90%, 95%, 96%, 97%, 98%, 99% or 100% identical to the amino acid sequence set forth in SEQ ID NO: 75; and ii) a VP2/VP3 capsid protein comprising an amino acid sequence at least 85%, 90%, 95%, 96%, 97%, 98%, 99% or 100% identical to the amino acid sequence set forth in SEQ ID NO: 124.

In some aspects, the capsid comprises: i) a VP1 capsid protein comprising an amino acid sequence at least 85%, 90%, 95%, 96%, 97%, 98%, 99% or 100% identical to the amino acid sequence set forth in SEQ ID NO: 245; and ii) a VP2/VP3 capsid protein comprising an amino acid sequence at least 85%, 90%, 95%, 96%, 97%, 98%, 99% or 100% identical to the amino acid sequence set forth in SEQ ID NO: 258.

In some aspects, the capsid comprises: i) a VP1 capsid protein comprising an amino acid sequence at least 85%, 90%, 95%, 96%, 97%, 98%, 99% or 100% identical to the amino acid sequence set forth in SEQ ID NO: 249; and ii) a VP2/VP3 capsid protein comprising an amino acid sequence at least 85%, 90%, 95%, 96%, 97%, 98%, 99% or 100% identical to the amino acid sequence set forth in SEQ ID NO: 262.

In some aspects, the capsid comprises: i) a VP1 capsid protein comprising an amino acid sequence at least 85%, 90%, 95%, 96%, 97%, 98%, 99% or 100% identical to the amino acid sequence set forth in SEQ ID NO: 255; and ii) a VP2/VP3 capsid protein comprising an amino acid sequence at least 85%, 90%, 95%, 96%, 97%, 98%, 99% or 100% identical to the amino acid sequence set forth in SEQ ID NO: 267.

The disclosure provides a vector comprising a nucleic acid sequence encoding a modified AAV capsid protein of the disclosure.

In some aspects, the nucleic acid sequence encoding a modified AAV capsid protein is operably linked to regulatory element that controls expression of the capsid protein in a host cell.

The disclosure provides an AAV viral vector comprising a modified capsid protein of the disclosure or an AAV capsid of the disclosure.

In some aspects, the AAV viral vector comprises a recombinant AAV (rAAV) vector encoding a therapeutic transgene or nucleotide sequence of interest (NOI).

The disclosure provides a cell comprising a vector of the disclosure or the AAV viral vector of the disclosure.

The disclosure provides a pharmaceutical composition comprising an AAV viral vector of claim the disclosure and at least one pharmaceutically acceptable excipient and/or additive.

The disclosure provides a method of providing a therapeutic transgene or protein to a subject, comprising administering to the subject an AAV viral vector of the disclosure or a pharmaceutical composition of the disclosure.

The disclosure provides a method of treating a subject having a disease and/or disorder, the method comprising administering to the subject at least one therapeutically effective amount of an AAV viral vector of the disclosure or a pharmaceutical composition of the disclosure. In some aspects, the disease and/or disorder is a muscular and/or neuromuscular disorder. In some aspects, the muscular and/or neuromuscular disorder is muscular dystrophy or myotonic dystrophy.

In some aspects, the AAV viral vector or the pharmaceutical composition is administered to the subject intravenously, intrathecally, intracerebrally, intraventricularly, intranasally, intratracheally, intra-aurally, intra-ocularly, or peri-ocularly, orally, rectally, transmucosally, inhalationally, transdermally, parenterally, subcutaneously, intradermally, intramuscularly, intracisternally, intranervally, intrapleurally, topically, intralymphatically, intracisternally or intranerve.

### BRIEF DESCRIPTION OF THE DRAWINGS

FIG. 1 is a schematic listing modified muscle-targeted AAV7 and AAVRh74 chimeric capsid proteins.

FIG. 2 is a schematic depicting muscle-targeted AAV Rh8 variant capsids comprising liver de-targeting amino acid mutations.

FIG. 3 is a schematic depicting modified muscle-targeted AAV capsid proteins of the disclosure.

FIG. 4 is a schematic depicting modified muscle-targeted AAVRh74 capsid proteins of the disclosure.

FIG. 5 shows immunofluorescence images showing transduction efficiency of HEK293 cells with an AAV vector comprising a VP1-specifically displayed targeting peptide.

FIGS. 6A-6D are immunofluorescence images showing enhanced transduction efficiency of C2C12 cells with modified muscle-targeted AAV capsids.

FIGS. 7A-C shows results of ex vivo imaging of AAV9, LBV30, and LBV31. FIG. 7A is an ex vivo immunofluorescence image showing in vivo targeting of AAV9, LBV30, and LBV31 capsids in mice. FIGS. 7B and 7C are graphs quantifying the immunofluorescence in various tissues.

FIG. 8 is a series of schematics depicting RGD peptide insertion scaffolds and approaches.

FIG. 9 is a graph depicting transduction of AAV viral vectors of the disclosure in the indicated mouse tissue: liver, heart, lung, spleen, kidney, intestine, testis, tongue, quadricep (quad), gastroenemius (gc), tibialis anterior (ta), diaphragm, and brain. The y-axis displays luminescence of an encapsidated luciferase reporter vector.

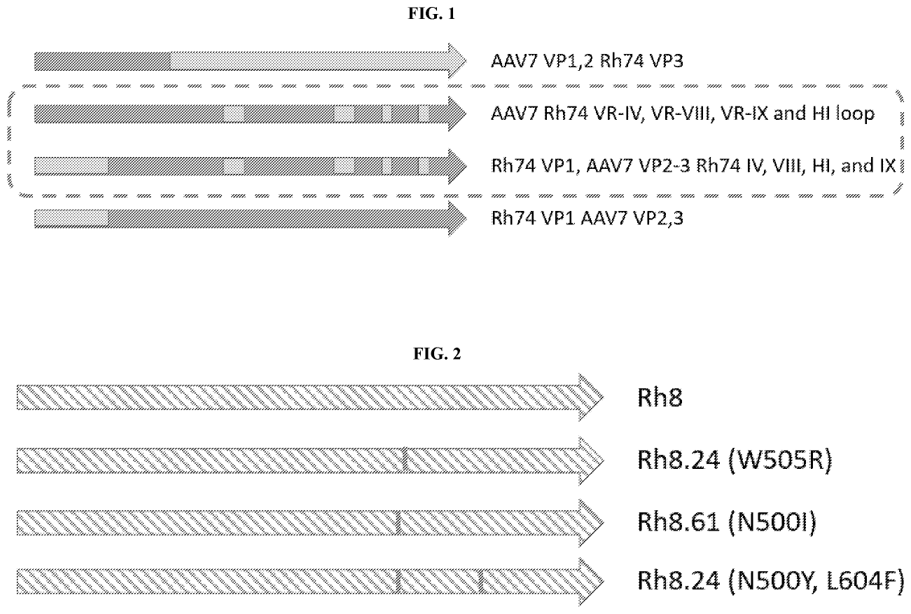

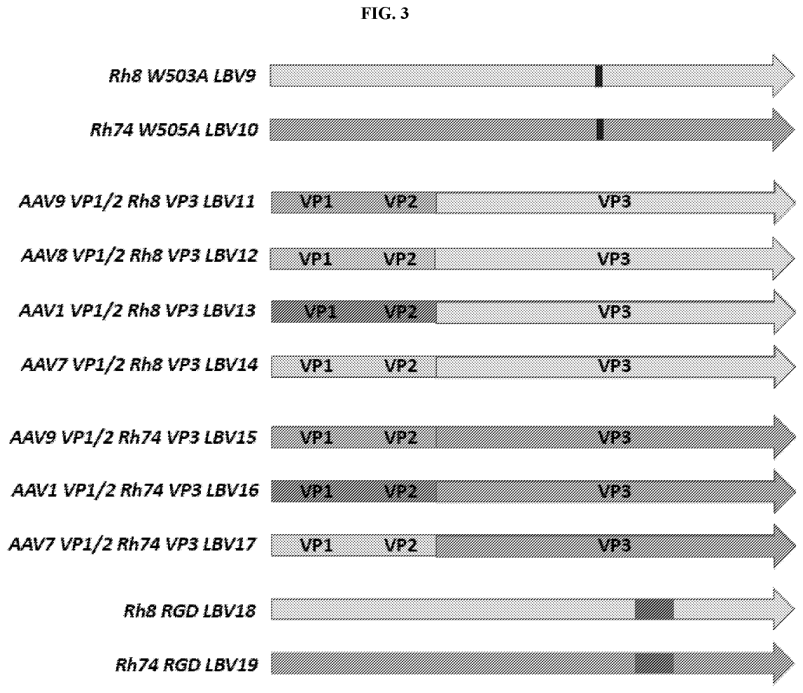

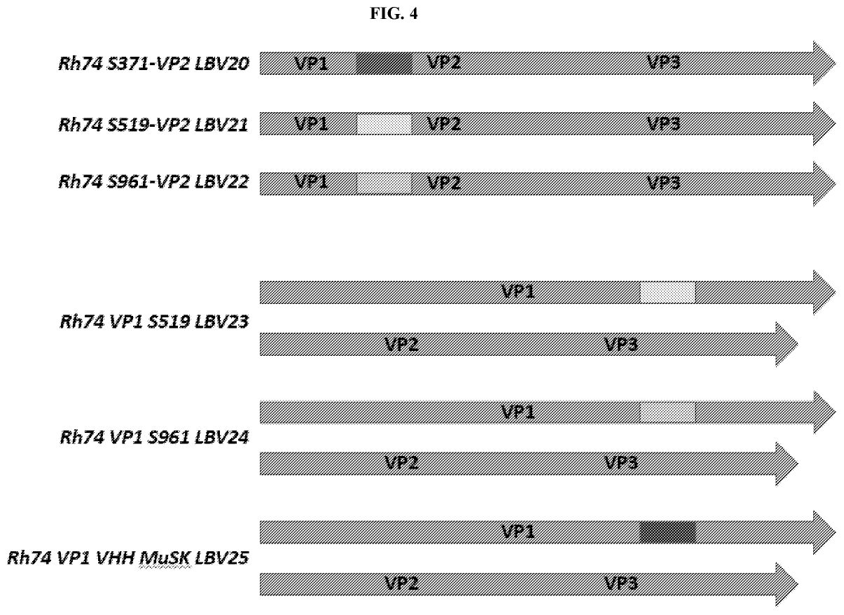

### DETAILED DESCRIPTION

The disclosure provides gene therapy compositions comprising modified and chimeric AAV capsid proteins for delivery of packaged therapeutics to muscular tissue.

The term “adeno-associated virus” or “AAV” as used herein refers to a member of the class of viruses associated with this name and belonging to the genus Dependoparvovirus, family Parvoviridae. Adeno-associated virus is a single-stranded DNA virus that grows in cells in which certain functions are provided by a co-infecting helper virus. General information and reviews of AAV can be found in, for example, Carter, 1989, Handbook of Parvoviruses, Vol. 1, pp. 169-228, and Berns, 1990, Virology, pp. 1743-1764, Raven Press, (New York). It is fully expected that the same principles described in these reviews will be applicable to additional AAV serotypes characterized after the publication dates of the reviews because it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3: 1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control. Multiple serotypes of this virus are known to be suitable for gene delivery; all known serotypes can infect cells from various tissue types. At least 11 sequentially numbered AAV serotypes are known in the art. Non-limiting exemplary serotypes useful in the methods disclosed herein include any of the 11 serotypes, e.g., AAV2, AAV8, AAV9, or variant serotypes, e.g., AAV-DJ and AAV PHP.B. The AAV particle comprises, consists essentially of, or consists of three major viral proteins: VP1, VP2 and VP3. In some aspects, the AAV refers to the serotype AAV1, AAV2, AAV4, AAV5, AAV6, AAV7, AAV8, AAV9, AAV10, AAV11, AAV12, AAV13, AAVPO1, AAVPHP.B, AAVrh74 or AAVrh.10.

Exemplary adeno-associated viruses and recombinant adeno-associated viruses include, but are not limited to all serotypes (e.g., AAV1, AAV2, AAV3, AAV4, AAV5, AAV6, AAV7, AAV8, AAV9, AAV10, AAV 11, AAV12, AAV13, AAVPO1, AAVPHP.B, AAVrh74 and AAVrh.10). Exemplary adeno-associated viruses and recombinant adeno-associated viruses include, but are not limited to, self-complementary AAV (scAAV) and AAV hybrids containing the genome of one serotype and the capsid of another serotype (e.g., AAV2/5, AAV-DJ and AAV-DJ8). Exemplary adeno-associated viruses and recombinant adeno-associated viruses include, but are not limited to, rAAV-LK03, AAV-KP-1 (described in detail in Kerun et al. JCI Insight, 2019; 4(22):e131610) and AAV-NP59 (described in detail in Paulk et al. Molecular Therapy, 2018; 26(1): 289-303).

### AAV Structure and Function

AAV is a replication-deficient parvovirus, the single-stranded DNA genome of which is about 4.7 kb in length, including two 145-nucleotide inverted terminal repeat (ITRs). There are multiple serotypes of AAV. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_001862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). The sequence of the AAV rh.74 genome is provided in U.S. Pat. No. 9,434,928. U.S. Pat. No. 9,434,928 also provides the sequences of the capsid proteins and a self-complementary genome. In one aspect, an AAV genome is a self-complementary genome. Cis-acting sequences directing viral DNA replication (rep), encapsidation/packaging, and host cell chromosome integration are contained within AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome.

The cap gene is expressed from the p40 promoter and encodes the three capsid proteins, VP1, VP2, and VP3. Alternative splicing and non-consensus translational start sites are responsible for the production of the three related capsid proteins. More specifically, after the single mRNA from which each of the VP1, VP2 and VP3 proteins are translated is transcribed, it can be spliced in two different manners: either a longer or shorter intron can be excised, resulting in the formation of two pools of mRNAs: a 2.3 kb- and a 2.6 kb-long mRNA pool. The longer intron is often preferred and thus the 2.3-kb-long mRNA can be called the major splice variant. This form lacks the first AUG codon, from which the synthesis of VP1 protein starts, resulting in a reduced overall level of VP1 protein synthesis. The first AUG codon that remains in the major splice variant is the initiation codon for the VP3 protein. However, upstream of that codon in the same open reading frame lies an ACG sequence (encoding threonine) which is surrounded by an optimal Kozak (translation initiation) context. This contributes to a low level of synthesis of the VP2 protein, which is actually the VP3 protein with additional N terminal residues, as is VP1, as described in Becerra S P et al., (December 1985). “Direct mapping of adeno-associated virus capsid proteins B and C: a possible ACG initiation codon”. Proceedings of the National Academy of Sciences of the United States of America. 82 (23): 7919-23, Cassinotti P et al., (November 1988). “Organization of the adeno-associated virus (AAV) capsid gene: mapping of a minor spliced mRNA coding for virus capsid protein 1”. Virology. 167 (1): 176-84, Muralidhar S et al., (January 1994). “Site-directed mutagenesis of adeno-associated virus type 2 structural protein initiation codons: effects on regulation of synthesis and biological activity”. Journal of Virology. 68 (1): 170-6, and Trempe J P, Carter B J (September 1988). “Alternate mRNA splicing is required for synthesis of adeno-associated virus VP1 capsid protein”. Journal of Virology. 62 (9): 3356-63, each of which is herein incorporated by reference. A single consensus polyA site is located at map position 95 of the AAV genome. The life cycle and genetics of AAV are reviewed in Muzyczka, Current Topics in Microbiology and Immunology, 158: 97-129 (1992).

Each VP1 protein contains a VP1 portion, a VP2 portion and a VP3 portion. The VP1 portion is the N-terminal portion of the VP1 protein that is unique to the VP1 protein. The VP2 portion is the amino acid sequence present within the VP1 protein that is also found in the N-terminal portion of the VP2 protein. The VP3 portion and the VP3 protein have the same sequence. The VP3 portion is the C-terminal portion of the VP1 protein that is shared with the VP1 and VP2 proteins.

The VP3 protein can be further divided into discrete variable surface regions I-IX (VRI-IX also referred to as VR1-VR8). Each of the variable surface regions (VRs) can comprise or contain specific amino acid sequences that either alone or in combination with the specific amino acid sequences of each of the other VRs can confer unique infection phenotypes (e.g., decreased antigenicity, improved transduction and/or tissue-specific tropism relative to other AAV serotypes) to a particular serotype as described in DiMatta et al., “Structural Insight into the Unique Properties of Adeno-Associated Virus Serotype 9” J. Virol., Vol. 86 (12): 6947-6958, June 2012, the contents of which are incorporated herein by reference.

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA to generate AAV vectors. The rep and cap proteins may be provided in trans. Another significant feature of AAV is that it is an extremely stable and hearty virus. It easily withstands the conditions used to inactivate adenovirus (560 to 65° C. for several hours), making cold preservation of AAV less critical. AAV may even be lyophilized. Finally, AAV-infected cells are not resistant to superinfection.

Multiple studies have demonstrated long-term (>1.5 years) recombinant AAV-mediated protein expression in muscle. See, Clark et al., Hum Gene Ther, 8: 659-669 (1997); Kessler et al., Proc Nat. Acad Sc. USA, 93: 14082-14087 (1996); and Xiao et al., J Virol, 70: 8098-8108 (1996). See also, Chao et al., Mol Ther, 2:619-623 (2000) and Chao et al., Mol Ther, 4:217-222 (2001). Moreover, because muscle is highly vascularized, recombinant AAV transduction has resulted in the appearance of transgene products in the systemic circulation following intramuscular injection as described in Herzog et al., Proc Natl Acad Sci USA, 94: 5804-5809 (1997) and Murphy et al., Proc Natl Acad Sci USA, 94: 13921-13926 (1997). Moreover, Lewis et al., J Virol, 76: 8769-8775 (2002) demonstrated that skeletal myofibers possess the necessary cellular factors for correct antibody glycosylation, folding, and secretion, indicating that muscle is capable of stable expression of secreted protein therapeutics. Recombinant AAV (rAAV) genomes of the invention comprise, consist essentially of, or consist of a nucleic acid molecule encoding a therapeutic protein and one or more AAV ITRs flanking the nucleic acid molecule. Production of pseudotyped rAAV is disclosed in, for example, WO2001083692. Other types of rAAV variants, for example rAAV with capsid mutations, are also contemplated. See, e.g., Marsic et al., Molecular Therapy, 22(11): 1900-1909 (2014). The nucleotide sequences of the genomes of various AAV serotypes are known in the art.

### Recombinant AAV Vectors

An “rAAV vector” as used herein refers to a vector comprising, consisting essentially of, or consisting of one or more transgene sequences and one or more AAV inverted terminal repeat sequences (ITRs). Such AAV vectors can be replicated and packaged into infectious viral particles, comprising modified AAV capsid proteins of the disclosure, when present in a host cell that provides the functionality of rep and cap gene products; for example, by transfection of the host cell. In some aspects, AAV vectors contain a promoter, at least one nucleic acid that may encode at least one protein or RNA, and/or an enhancer and/or a terminator within the flanking ITRs that is packaged into the infectious AAV particle. The encapsidated nucleic acid portion may be referred to as the AAV vector genome. Plasmids containing rAAV vectors may also contain elements for manufacturing purposes, e.g., antibiotic resistance genes, origin of replication sequences etc., but these are not encapsidated and thus do not form part of the AAV particle.

In some aspects, an rAAV vector can comprise at least one transgene nucleic acid molecule. In some aspects, an rAAV vector can comprise at least one AAV inverted terminal (ITR) sequence. In some aspects, an rAAV vector can comprise at least one promoter sequence. In some aspects, an rAAV vector can comprise at least one enhancer sequence. In some aspects, an rAAV vector can comprise at least one polyA sequence. In some aspects, an rAAV vector can comprise at least one reporter protein.

In some aspects, an rAAV vector can comprise a first AAV ITR sequence, a promoter sequence, a transgene nucleic acid molecule, a polyA sequence, and a second AAV ITR sequence. In some aspects, an rAAV vector can comprise, in the 5′ to 3′ direction, a first AAV ITR sequence, a promoter sequence, a transgene nucleic acid molecule, a polyA sequence, and a second AAV ITR sequence.

In some aspects, an rAAV vector can comprise more than one transgene nucleic acid molecule. In some aspects, an rAAV vector can comprise at least two transgene nucleic acid molecules, such that the rAAV vector comprises a first transgene nucleic acid molecule and an at least second transgene nucleic acid molecule. In some aspects, the first and the at least second transgene nucleic acid molecule can comprise the same nucleic acid sequence. In some aspects, the first and the at least second transgene nucleic acid molecules can comprise different nucleic acid sequences. In some aspects, the first and the at least second transgene nucleic acid sequences can be adjacent to each other.

In some aspects, an rAAV vector can comprise more than one promoter sequence. In some aspects, an rAAV vector can comprise at least two promoter sequences, such that the rAAV vector comprises a first promoter sequence and an at least second promoter sequence. In some aspects, the first and the at least second promoter sequences can comprise the same sequence. In some aspects, the first and the at least second promoter sequences can comprise different sequences. In some aspects, the first and the at least second promoter sequences can be adjacent to each other. In some aspects wherein an rAAV vector also comprises a first transgene nucleic acid molecule and an at least second transgene nucleic acid molecule, the first promoter can be located upstream (5′) of the first transgene nucleic acid molecule and the at least second promoter can be located between the first transgene nucleic acid molecule and the at least second transgene nucleic acid molecule, such that the at least second promoter is downstream (3′) of the first transgene nucleic acid molecule and upstream (5′) of the at least second transgene nucleic acid molecule.

Any of the preceding rAAV vectors can further comprise at least one enhancer. The at least one enhancer can be located anywhere in the rAAV vector. In some aspects, the at least one enhancer can be located immediately upstream (5′) of a promoter. Thus, an rAAV vector can comprise, in the 5′ to 3′ direction, a first AAV ITR sequence, an enhancer, a promoter sequence, a transgene nucleic acid molecule, a polyA sequence, and a second AAV ITR sequence. In some aspects, the at least one enhancer can be located immediately downstream (3′) of a promoter. Thus, an rAAV vector can comprise, in the 5′ to 3′ direction, a first AAV ITR sequence, a promoter sequence, an enhancer, a transgene nucleic acid molecule, a polyA sequence, and a second AAV ITR sequence. In some aspects, the at least one enhancer can be located immediately downstream of a transgene nucleic acid molecule. Thus, an rAAV vector can comprise, in the 5′ to 3′ direction, a first AAV ITR sequence, a promoter sequence, a transgene nucleic acid molecule, an enhancer, a polyA sequence, and a second AAV ITR sequence.

rAAV vectors of the disclosure can comprise any transgene nucleic acid molecule known in the art. In some aspects, the transgene nucleic acid is a therapeutic transgene. In some aspects, a transgene nucleic acid molecule is referred to interchangeable as a nucleotide sequence of interest (NOI). An NOI includes, without limitation, any nucleotide sequence or transgene capable of being delivered by a vector. NOIs can be synthetic, derived from naturally occurring DNA or RNA, codon optimized, recombinant RNA/DNA, cDNA, partial genomic DNA, and/or combinations thereof. The NOI can be a coding region or partial coding region, but need not be a coding region. An NOI can be RNA/DNA in a sense or anti-sense orientation. NOIs are also referred herein, without limitation, as transgenes, heterologous sequences, genes, therapeutic genes. An NOI may also encode a POI (protein of interest), a partial POI, a mutated version or variant of a POI. A POI may be analogous to or correspond to a wild-type protein. A POI may also be a fusion protein or nucleoprotein complex such as a CRISPR/Cas nucleoprotein complex. A POI may also be a PUF or PUMBY protein. In some aspects, POIs can be RNA targeting or RNA-binding proteins or nucleoprotein complexes.

### NOIs or Transgenes Comprising Non-Guided RNA-Binding Fusion Proteins

In some embodiments, the NOI is a nucleic acid encoding a target RNA-binding fusion protein which is not an RNA-guided target RNA-binding fusion protein and as such comprises at least one RNA-binding polypeptide which is capable of binding a target RNA without a corresponding gRNA sequence. Such non-guided RNA-binding polypeptides include, without limitation, at least one RNA-binding protein or RNA-binding portion thereof which is a PUF (Pumilio and FBF homology family) protein. This type RNA-binding polypeptide can be used instead of a gRNA-guided RNA binding protein such as CRISPR/Cas. The unique RNA recognition mode of PUF proteins (named for Drosophila Pumilio and C. elegans fem-3 binding factor) that are involved in mediating mRNA stability and translation are well known in the art. The PUF domain of human Pumilio1, also known in the art, binds tightly to cognate RNA sequences and its specificity can be modified. It contains eight PUF modules that recognize eight consecutive RNA bases with each module recognizing a single base. Since two amino acid side chains in each module recognize the Watson-Crick edge of the corresponding base and determine the specificity of that module, a PUF protein can be designed to specifically bind most 8 to 16-nt RNA. Wang et al., Nat Methods. 2009; 6(11): 825-830. See also WO2012/068627 which is incorporated by reference herein in its entirety.

The modular nature of the PUF-RNA interaction has been used to rationally engineer the binding specificity of PUF domains (Cheong, C. G. & Hall, T. M. (2006) PNAS 103: 13635-13639; Wang, X. et al (2002) Cell 110: 501-512). However, only the successful design of PUF proteins with modules that recognize adenine, guanine or uracil have been reported prior to the teachings of WO2012/06827 supra. While the wild-type PumHD does not bind cytosine (C), molecular engineering has shown that some of the Pum units can be mutated to bind C with good yield and specificity. See e.g., Dong, S. et al. Specific and modular binding code for cytosine recognition in Pumilio/FBF (PUF) RNA-binding domains, The Journal of biological chemistry 286, 26732-26742 (2011). Accordingly, PumHD is a modified version of the WT Pumilio protein that exhibits programmable binding to arbitrary 8-base sequences of RNA. Each of the eight units of PumHD can bind to all four RNA bases, and the RNA bases flanking the target sequence do not affect binding. See also the following for art-recognized RNA-binding rules of PUF design: Filipovska A, Razif M F, Nygird K K, & Rackham O. A universal code for RNA recognition by PUF proteins. Nature chemical biology, 7(7), 425-427 (2011); Filipovska A, & Rackham O. Modular recognition of nucleic acids by PUF, TALE and PPR proteins. Molecular BioSystems, 8(3), 699-708 (2012); Abil Z, Denard C A, & Zhao H. Modular assembly of designer PUF proteins for specific post-transcriptional regulation of endogenous RNA. Journal of biological engineering, 8(1), 7 (2014); Zhao Y, Mao M, Zhang W, Wang J, Li H, Yang Y, Wang Z, & Wu J. Expanding RNA binding specificity and affinity of engineered PUF domains. Nucleic Acids Research, 46(9), 4771-4782 (2018); Shinoda K, Tsuji S, Futaki S, & Imanishi M. Nested PUF Proteins Extending Target RNA Elements for Gene Regulation. ChemBioChem, 19(2), 171-176 (2018); Koh Y Y, Wang Y, Qiu C, Opperman L, Gross L, Tanaka Hall T M, & Wickens M. Stacking Interactions in PUF-RNA Complexes. RNA, 17(4), 718-727 (2011).

As such, it is well known in the art that human PUM1 (1186 amino acids) contains an RNA-binding domain (RBD) in the C-terminus of the protein (also known as Pumilio homology domain PUM-HD amino acid 828-amino acid 1175) and that PUFs are based on the RBD of human PUM1. There are 8 structural repeat modules of 36 amino acids (except module 7 which has 43 amino acids) for RNA binding and flanking N- and C-terminal regions important for protein structure and stability. Within each repeat module, amino acids 12, 13, and 16 are important for RNA binding with 12 and 16 responsible for RNA base recognition. Amino acid 13 stacks with RNA bases and can be modified to tune specificity and affinity. Alternatively, the PUF design may maintain amino acid 13 as human PUM1's native residue. In some embodiments of the PUF or PUMBY compositions disclosed herein, amino acid 13 (for stacking) will be engineered with an H and in other embodiments, will be engineered with a Y. In some embodiments, stacking residues may be modified to improve binding and specificity. Recognition occurs in reverse orientation as N- to C-terminal PUF recognizes 3′ to 5′ RNA. Accordingly, PUF engineering of 8 modules (8PUF), as known in the art, mimics a human protein. An exemplary 8-mer RNA recognition (8PUF) would be designed as follows: R1′-R1-R2-R3-R4-R5-R6-R7-R8-R8′. In one embodiment, an 8PUF is used as the RBD. In another embodiment, a variation of the 8PUF design is used to create a 14-mer RNA recognition (14PUF) RBD, 15-mer RNA recognition (15PUF) RBD, or a 16-mer RNA recognition (16PUF) RBD. In another embodiment, the PUF can be engineered to comprise a 4-mer, 5-mer, 6-mer, 7-mer, 8-mer, 9-mer, 10-mer, 11-mer, 12-mer, 13-mer, 14-mer, 15-mer, 16-mer, 24-mer, 30-mer, 36-mer, or any number of modules between. Shinoda et al., 2018; Criscuolo et al., 2020 See also U.S. Pat. No. 9,580,714 which is incorporated herein in its entirety.

In some embodiments of the non-guided RNA-binding fusion proteins of the disclosure, the fusion protein comprises at least one RNA-binding protein or RNA-binding portion thereof which is a PUMBY (Pumilio-based assembly) protein. RNA-binding protein PumHD, which has been widely used in native and modified form for targeting RNA, has been engineered into a protein architecture designed to yield a set of four canonical protein modules, each of which targets one RNA base. These modules (i.e., Pumby, for Pumilio-based assembly) are concatenated in chains of varying composition and length, to bind desired target RNAs. In essence, PUMBY is a more simple and modular form of PumHD, in which a single protein unit of PumHD is concatenated into arrays of arbitrary size and binding sequence specificity. The specificity of such Pumby-RNA interactions is high, with undetectable binding of a Pumby chain to RNA sequences that bear three or more mismatches from the target sequence. Katarzyna et al., PNAS, 2016; 113(19): E2579-E2588. See also US 2016/0238593 which is incorporated by reference herein in its entirety.

In some embodiments of the compositions of the disclosure, the first RNA binding protein comprises a Pumilio and FBF (PUF) protein. In some embodiments, the first RNA binding protein comprises a Pumilio-based assembly (PUMBY) protein. In some embodiments, the PUF or PUMBY RNA-binding proteins are fused with a nuclease domain such as is an zinc-finger endonuclease known as ZC3H12A (E17).

In some embodiments of the compositions of the disclosure, at least one of the RNA-binding proteins or RNA-binding portions thereof is a PPR protein. PPR proteins (proteins with pentatricopeptide repeat (PPR) motifs derived from plants) are nuclear-encoded and exclusively controlled at the RNA level organelles (chloroplasts and mitochondria), cutting, translation, splicing, RNA editing, genes specifically acting on RNA stability. PPR proteins are typically a motif of 35 amino acids and have a structure in which a PPR motif is about 10 contiguous amino acids. The combination of PPR motifs can be used for sequence-selective binding to RNA. PPR proteins are often comprised of PPR motifs of about 10 repeat domains. PPR domains or RNA-binding domains may be configured to be catalytically inactive. WO 2013/058404 incorporated herein by reference in its entirety.

In some embodiments, the fusion protein disclosed herein comprises a linker between the at least two RNA-binding polypeptides. In some embodiments, the linker is a peptide linker. In some embodiments, the peptide linker comprises one or more repeats of the tri-peptide GGS. In other embodiments, the linker is a non-peptide linker. In some embodiments, the non-peptide linker comprises polyethylene glycol (PEG), polypropylene glycol (PPG), co-poly(ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacryl amide, polyacrylate, polycyanoacrylates, lipid polymers, chitins, hyaluronic acid, heparin, or an alkyl linker.

In some embodiments, the at least one RNA-binding protein does not require multimerization for RNA-binding activity. In some embodiments, the at least one RNA-binding protein is not a monomer of a multimer complex. In some embodiments, a multimer protein complex does not comprise the RNA binding protein. In some embodiments, the at least one of RNA-binding protein selectively binds to a target sequence within the RNA molecule. In some embodiments, the at least one RNA-binding protein does not comprise an affinity for a second sequence within the RNA molecule. In some embodiments, the at least one RNA-binding protein does not comprise a high affinity for or selectively bind a second sequence within the RNA molecule. In some embodiments, the at least one RNA-binding protein comprises between 2 and 1300 amino acids, inclusive of the endpoints.

In some embodiments, the at least one RNA-binding protein of the fusion proteins disclosed herein further comprises a sequence encoding a nuclear localization signal (NLS). In some embodiments, a nuclear localization signal (NLS) is positioned at the N-terminus of the RNA binding protein. In some embodiments, the at least one RNA-binding protein comprises an NLS at a C-terminus of the protein. In some embodiments, the at least one RNA-binding protein further comprises a first sequence encoding a first NLS and a second sequence encoding a second NLS. In some embodiments, the first NLS or the second NLS is positioned at the N-terminus of the RNA-binding protein. In some embodiments, the at least one RNA-binding protein comprises the first NLS or the second NLS at a C-terminus of the protein. In some embodiments, the at least one RNA-binding protein further comprises an NES (nuclear export signal) or other peptide tag or secretory signal. In one embodiment, the tag is a FLAG tag.

In some embodiments, a fusion protein disclosed herein comprises the at least one RNA-binding protein as a first RNA-binding protein together with a second RNA-binding protein comprising or consisting of a nuclease domain.

In some embodiments, the second RNA-binding polypeptide is operably configured to the first RNA-binding polypeptide at the C-terminus of the first RNA-binding polypeptide. In some embodiments, the second RNA-binding polypeptide is operably configured to the first RNA-binding polypeptide at the N-terminus of the first RNA-binding polypeptide. In one embodiment, an exemplary fusion protein is a PUF or PUMBY-based first RNA-binding protein fused to a second RNA-binding protein which is an zinc-finger endonuclease known as ZC3H12A.

### NOIs or Transgenes Comprising Guide RNAs for RNA-Guided RNA-Binding Proteins

In a Cas-based RNA-targeting gene therapy system, an NOI or transgene comprises a guide RNA.

The terms guide RNA (gRNA) and single guide RNA (sgRNA) are used interchangeably throughout the disclosure.

Guide RNAs (gRNAs) of the disclosure may comprise of a spacer sequence and a “direct repeat” (DR) sequence. In some embodiments, a guide RNA is a single guide RNA (sgRNA) comprising a contiguous spacer sequence and DR sequence. In some embodiments, the spacer sequence and the DR sequence are not contiguous. In some embodiments, the gRNA comprises a DR sequence. DR sequences refer to the repetitive sequences in the CRISPR locus (naturally-occurring in a bacterial genome or plasmid) that are interspersed with the spacer sequences. It is well known that one would be able to infer the DR sequence of a corresponding (or cognate) Cas protein if the sequence of the associated CRISPR locus is known. In some embodiments, a guide RNA comprises a direct repeat (DR) sequence and a spacer sequence. In some embodiments, a sequence encoding a guide RNA or single guide RNA of the disclosure comprises or consists of a spacer sequence and a DR sequence, that are separated by a linker sequence. In some embodiments, the linker sequence may comprise or consist of 1, 2, 3, 4, 5, 6, 7, 8, 9, 10, 15, 20, 25, 30, 35, 40, 45, 50 or any number of nucleotides (nt) in between. In some embodiments, the linker sequence may comprise at least 1, 2, 3, 4, 5, 6, 7, 8, 9, 10, 15, 20, 25, 30, 35, 40, 45, 50 or any number of nucleotides in between. In some embodiments, the DR sequence is a Cas13d DR sequence.

In one embodiment, the gRNA that hybridizes with the one or more target RNA molecules in a Cas 13d-mediated manner includes one or more direct repeat (DR) sequences, one or more spacer sequences, such as, e.g., one or more sequences comprising an array of DR-spacer-DR-spacer. In one embodiment, a plurality of gRNAs are generated from a single array, wherein each gRNA can be different, for example target different RNAs or target multiple regions of a single RNA, or combinations thereof. In some embodiments, an isolated gRNA includes one or more direct repeat sequences, such as an unprocessed (e.g., about 36 nt) or processed DR (e.g., about 30 nt). In some embodiments, a gRNA can further include one or more spacer sequences specific for (e.g., is complementary to) the target RNA. In certain such embodiments, multiple polIII promoters can be used to drive multiple gRNAs, spacers and/or DRs. In one embodiment, a guide array comprises a DR (about 36 nt)-spacer (about 30 nt)-DR (about 36 nt)-spacer (about 30 nt).

Guide RNAs (gRNAs) of the disclosure may comprise non-naturally occurring nucleotides. In some embodiments, a guide RNA of the disclosure or a sequence encoding the guide RNA comprises or consists of modified or synthetic RNA nucleotides. Exemplary modified RNA nucleotides include, but are not limited to, pseudouridine (Ψ), dihydrouridine (D), inosine (I), and 7-methylguanosine (m7G), hypoxanthine, xanthine, xanthosine, 7-methylguanine, 5, 6-Dihydrouracil, 5-methylcytosine, 5-methylcytidine, 5-hydropxymethylcytosine, isoguanine, and isocytosine.

Guide RNAs (gRNAs) of the disclosure may bind modified RNA within a target sequence. Within a target sequence, guide RNAs (gRNAs) of the disclosure may bind modified or mutated (e.g., pathogenic) RNA. Exemplary epigenetically or post-transcriptionally modified RNA include, but are not limited to, 2′-O-Methylation (2′-OMe) (2′-O-methylation occurs on the oxygen of the free 2′-OH of the ribose moiety), N6-methyladenosine (m6A), and 5-methylcytosine (m5C).

In some embodiments of the compositions of the disclosure, a guide RNA of the disclosure comprises at least one sequence encoding a non-coding C/D box small nucleolar RNA (snoRNA) sequence. In some embodiments, the snoRNA sequence comprises at least one sequence that is complementary to the target RNA, wherein the target sequence of the RNA molecule comprises at least one 2′-OMe. In some embodiments, the snoRNA sequence comprises at least one sequence that is complementary to the target RNA, wherein the at least one sequence that is complementary to the target RNA comprises a box C motif (RUGAUGA) and a box D motif (CUGA).

Spacer sequences of the disclosure bind to the target sequence of an RNA molecule. In some embodiments, spacer sequences of the disclosure bind to pathogenic target RNA.

In some embodiments of the compositions of the disclosure, the sequence comprising the gRNA further comprises a spacer sequence that specifically binds to the target RNA sequence. In some embodiments, the spacer sequence has at least 50%, 55%, 60%, 65%, 70%, 75%, 80%, 87%, 90%, 95%, 97%, 99% or any percentage in between of complementarity to the target RNA sequence. In some embodiments, the spacer sequence has 100% complementarity to the target RNA sequence. In some embodiments, the spacer sequence comprises or consists of 20 nucleotides. In some embodiments, the spacer sequence comprises or consists of 21 nucleotides, 22 nucleotides, 23 nucleotides, 24 nucleotides, 25 nucleotides, 26 nucleotides, 27 nucleotides, 28 nucleotides, or 29 nucleotides. In some embodiments, the spacer sequence comprises or consists of 26 nucleotides. In some embodiments, the spacer sequence is non-processed and comprises or consists of 30 nucleotides. In some embodiments the non-processed spacer sequence comprises or consists of 30-36 nucleotides.

DR sequences of the disclosure bind the Cas polypeptide of the disclosure. Upon binding of the spacer sequence of the gRNA to the target RNA sequence, the Cas protein bound to the DR sequence of the gRNA is positioned at the target RNA sequence. A DR sequence having sufficient complementarity to its cognate Cas protein, or nucleic acid thereof, binds selectively to the target nucleic acid sequence of the Cas protein and has at least 50%, 55%, 60%, 65%, 70%, 75%, 80%, 85%, 90%, 95%, 96, 97%, 98%, 99%, or any percentage identity in between to the sequence. In some embodiments, a sequence having sufficient complementarity has 100% identity. In some embodiments, DR sequences of the disclosure comprise a secondary structure or a tertiary structure. Exemplary secondary structures include, but are not limited to, a helix, a stem loop, a bulge, a tetraloop and a pseudoknot. Exemplary tertiary structures include, but are not limited to, an A-form of a helix, a B-form of a helix, and a Z-form of a helix. Exemplary tertiary structures include, but are not limited to, a twisted or helicized stem loop. Exemplary tertiary structures include, but are not limited to, a twisted or helicized pseudoknot. In some embodiments, DR sequences of the disclosure comprise at least one secondary structure or at least one tertiary structure. In some embodiments, DR sequences of the disclosure comprise one or more secondary structure(s) or one or more tertiary structure(s).

In some embodiments of the compositions of the disclosure, a guide RNA or a portion thereof selectively binds to a tetraloop motif in an RNA molecule of the disclosure. In some embodiments, a target sequence of an RNA molecule comprises a tetraloop motif. In some embodiments, the tetraloop motif is a “GRNA” motif comprising or consisting of one or more of the sequences of GAAA, GUGA, GCAA or GAGA.

In some embodiments of the compositions of the disclosure, a guide RNA or a portion thereof that binds to a target sequence of an RNA molecule hybridizes to the target sequence of the RNA molecule. In some embodiments, a guide RNA or a portion thereof that binds to a first RNA binding protein or to a second RNA binding protein covalently binds to the first RNA binding protein or to the second RNA binding protein. In some embodiments, a guide RNA or a portion thereof that binds to a first RNA binding protein or to a second RNA binding protein non-covalently binds to the first RNA binding protein or to the second RNA binding protein.

In some embodiments of the compositions of the disclosure, a guide RNA or a portion thereof comprises or consists of between 10 and 100 nucleotides, inclusive of the endpoints. In some embodiments, a spacer sequence of the disclosure comprises or consists of between 10 and 30 nucleotides, inclusive of the endpoints. In some embodiments, a spacer sequence of the disclosure comprises or consists of 15, 16, 17, 18, 19, 20, 21, 22, 23, 24, 25, 26, 27, 28, 29 or 30 nucleotides. In some embodiments, the spacer sequence of the disclosure comprises or consists of 20 nucleotides. In some embodiments, the spacer sequence of the disclosure comprises or consists of 21 nucleotides. In some embodiments, the spacer sequence of the disclosure comprises or consists of 26 nucleotides.

Guide molecules generally exist in various states of processing. In one example, an unprocessed guide RNA is 36 nt of DR followed by 30-32 nt of spacer. The guide RNA is processed (truncated/modified) by Cas 13d itself or other RNases into the shorter “mature” form. In some embodiments, an unprocessed guide sequence is about, or at least about 30, 35, 40, 45, 50, 55, 60, 61, 62, 63, 64, 65, 66, 67, 68, 69, 70, 71, 72, 73, 74, 75, or more nucleotides (nt) in length. In some embodiments, a processed guide sequence is about 44 to 60 nt (such as 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 nt). In some embodiments, an unprocessed spacer is about 28-32 nt long (such as 25, 26, 27, 28, 29, 30, 31, 32, 33, 34, or 35 nt) while the mature (processed) spacer can be about 10 to 30 nt, 10 to 25 nt, 14 to 25 nt, 20 to 22 nt, or 14-30 nt (such as 10, 11, 12, 13, 14, 15, 16, 17, 18, 19, 20, 21, 22, 23, 24, 25, 26, 27, 28, 29, 30, 31, 32, 33, 34, or 35 nt). In some embodiments, an unprocessed DR is about 36 nt (such as 30, 31, 32, 33, 34, 35, 36, 37, 38, 39, 40 or 41 nt), while the processed DR is about 30 nt (such as 25, 26, 27, 28, 29, 30, 31, 32, 33, 34, or 35 nt). In some embodiments, a DR sequence is truncated by 1-10 nucleotides (such as 1, 2, 3, 4, 5, 6, 7, 8, 9, to 10 nucleotides at e.g., the 5′ end in order to be expressed as mature pre-processed guide RNAs.

In some embodiments of the compositions of the disclosure, a guide RNA or a portion thereof does not comprise a nuclear localization sequence (NLS).

In some embodiments of the compositions of the disclosure, a guide RNA or a portion thereof comprises a sequence complementary to a protospacer flanking sequence (PFS). In some embodiments, including those wherein a guide RNA or a portion thereof comprises a sequence complementary to a PFS, the first RNA binding protein may comprise a sequence isolated or derived from a Cas13 protein. In some embodiments, including those wherein a guide RNA or a portion thereof comprises a sequence complementary to a PFS, the first RNA binding protein may comprise a sequence encoding a Cas13 protein or an RNA-binding portion thereof. In some embodiments, the guide RNA or a portion thereof does not comprise a sequence complementary to a PFS.

In some embodiments of the compositions of the disclosure, guide RNA sequence of the disclosure comprises a promoter sequence to drive expression of the guide RNA. In some embodiments, a vector comprising a guide RNA sequence of the disclosure comprises a promoter sequence to drive expression of the guide RNA. In some embodiments, the promoter to drive expression of the guide RNA is a constitutive promoter. In some embodiments, the promoter sequence is an inducible promoter. In some embodiments, the promoter is a sequence is a tissue-specific and/or cell-type specific promoter. In some embodiments, the promoter is a hybrid or a recombinant promoter. In some embodiments, the promoter is a promoter capable of expressing the guide RNA in a mammalian cell. In some embodiments, the promoter is a promoter capable of expressing the guide RNA in a human cell. In some embodiments, the promoter is a promoter capable of expressing the guide RNA and restricting the guide RNA to the nucleus of the cell. In some embodiments, the promoter is a human RNA polymerase promoter or a sequence isolated or derived from a sequence encoding a human RNA polymerase promoter. In some embodiments, the promoter is a U6 promoter or a sequence isolated or derived from a sequence encoding a U6 promoter. In some embodiments, the U6 promoter is a human U6 promoter. In some embodiments, the promoter is a human tRNA promoter or a sequence isolated or derived from a sequence encoding a human tRNA promoter. In some embodiments, the promoter is a human valine tRNA promoter or a sequence isolated or derived from a sequence encoding a human valine tRNA promoter.

In some embodiments of the compositions of the disclosure, a promoter to drive expression of the guide RNA further comprises a regulatory element. In some embodiments, a vector comprising a promoter sequence to drive expression of the guide RNA further comprises a regulatory element. In some embodiments, a regulatory element enhances expression of the guide RNA. Exemplary regulatory elements include, but are not limited to, an enhancer element, an intron, an exon, or a combination thereof.

In some embodiments of the compositions of the disclosure, a vector of the disclosure comprises one or more of a sequence encoding a guide RNA, a promoter sequence to drive expression of the guide RNA and a sequence encoding a regulatory element. In some embodiments of the compositions of the disclosure, the vector further comprises a sequence encoding a fusion protein of the disclosure.

### RNA-Guided RNA-Binding Proteins

In some embodiments of the compositions of the disclosure, gRNAs correspond to target RNA molecules and an RNA-guided RNA binding protein. In some embodiments, the gRNAs correspond to an RNA-guided RNA binding fusion protein, wherein the fusion protein comprises first and second RNA binding proteins. In some embodiments, the first RNA-binding protein in the fusion protein is a deactivated RNA-binding protein, e.g., a deactivated Cas or catalytic dead Cas protein. In some embodiments, along a sequence encoding the RNA-binding fusion protein, the sequence encoding the first RNA binding protein is positioned 5′ of the sequence encoding the second RNA binding protein. In some embodiments, along a sequence encoding the fusion protein, the sequence encoding the first RNA binding protein is positioned 3′ of the sequence encoding the second RNA binding protein.

In some embodiments of the compositions of the disclosure, the sequence encoding the first RNA binding protein comprises a sequence isolated or derived from a protein capable of binding an RNA molecule. In some embodiments, the sequence encoding the first RNA binding protein comprises a sequence isolated or derived from a protein capable of selectively binding an RNA molecule and not binding a DNA molecule, a mammalian DNA molecule or any DNA molecule. In some embodiments, the sequence encoding the first RNA binding protein comprises a sequence isolated or derived from a protein capable of binding an RNA molecule and inducing a break in the RNA molecule. In some embodiments, the sequence encoding the first RNA binding protein comprises a sequence isolated or derived from a protein capable of binding an RNA molecule, inducing a break in the RNA molecule, and not binding a DNA molecule, a mammalian DNA molecule or any DNA molecule. In some embodiments, the sequence encoding the first RNA binding protein comprises a sequence isolated or derived from a protein capable of binding an RNA molecule, inducing a break in the RNA molecule, and neither binding nor inducing a break in a DNA molecule, a mammalian DNA molecule or any DNA molecule.

In some embodiments of the compositions of the disclosure, the sequence encoding the first RNA-guided RNA binding protein comprises a sequence isolated or derived from a protein with no DNA nuclease activity.

In some embodiments of the compositions of the disclosure, the sequence encoding the RNA-guided RNA binding protein disclosed herein comprises a sequence isolated or derived from a CRISPR Cas protein. In some embodiments, the CRISPR Cas protein is not a Type II CRISPR Cas protein. In some embodiments, the CRISPR Cas protein is not a Cas9 protein. In some embodiments, the Cas9 protein is engineered to target RNA (RCas9). In some embodiments of the compositions of the disclosure, the sequence encoding the RNA-guided RNA binding protein comprises a Type VI CRISPR Cas protein or portion thereof. In some embodiments, the Type VI CRISPR Cas protein comprises a Cas13 protein or portion thereof. Exemplary Cas13 proteins of the disclosure may be isolated or derived from any species, including, but not limited to, a bacteria or an archaea. Exemplary Cas13 proteins of the disclosure may be isolated or derived from any species, including, but not limited to, Leptotrichia wadei, Listeria seeligeri serovar 112b (strain ATCC 35967 DSM 20751 CIP 100100 SLCC 3954), Lachnospiraceae bacterium, Clostridium aminophilum DSM 10710, Carnobacterium gallinarum DSM 4847, Paludibacter propionicigenes WB4, Listeria weihenstephanensis FSL R9-0317, Listeria weihenstephanensis FSL R9-0317, bacterium FSL M6-0635 (Listeria newyorkensis), Leptotrichia wadei F0279, Rhodobacter capsulatus SB 1003, Rhodobacter capsulatus R121, Rhodobacter capsulatus DE442 and Corynebacterium ulcerans. Exemplary Cas13 proteins of the disclosure may be DNA nuclease inactivated. Exemplary Cas13 proteins of the disclosure include, but are not limited to, Cas13a, Cas13b, Cas13c, Cas13d and orthologs thereof. Exemplary Cas13b proteins of the disclosure include, but are not limited to, subtypes 1 and 2 referred to herein as Csx27 and Csx28, respectively.

### AAV Viral Vectors

A “viral vector” is defined as a recombinantly produced virus or viral particle that contains a polynucleotide to be delivered into a host cell, either in vivo, ex vivo or in vitro. Examples of viral vectors include retroviral vectors, AAV vectors, lentiviral vectors, adenovirus vectors, alphavirus vectors and the like. Alphavirus vectors, such as Semliki Forest virus-based vectors and Sindbis virus-based vectors, have also been developed for use in gene therapy and immunotherapy. See, e.g., Schlesinger and Dubensky (1999) Curr. Opin. Biotechnol. 5:434-439 and Ying, et al. (1999) Nat. Med. 5(7):823-827.

An “AAV virion” or “AAV viral particle” or “AAV viral vector” or “rAAV viral vector” or “AAV vector particle” or “AAV particle” refers to a viral particle composed of at least one AAV capsid protein and an encapsidated polynucleotide rAAV vector. Thus, production of an rAAV viral vector necessarily includes production of an rAAV vector, as such a vector is contained within an rAAV vector.

As used herein, the term “viral capsid” or “capsid” refers to the proteinaceous shell or coat of a viral particle. Capsids function to encapsidate, protect, transport, and release into the host cell a viral genome. Capsids are generally comprised of oligomeric structural subunits of protein (“capsid proteins”). As used herein, the term “encapsidated” means enclosed within a viral capsid. The viral capsid of AAV is composed of a mixture of three viral capsid proteins: VP1, VP2, and VP3. The mixture of VP1, VP2 and VP3 contains 60 monomers that are arranged in a T=1 icosahedral symmetry in a ratio of 1:1:10 (VP1:VP2:VP3) or 1:1:20 (VP1:VP2:VP3) as described in Sonntag F et al., (June 2010). “A viral assembly factor promotes AAV2 capsid formation in the nucleolus”. Proceedings of the National Academy of Sciences of the United States of America. 107 (22): 10220-5, and Rabinowitz J E, Samulski R J (December 2000). “Building a better vector: the manipulation of AAV virions”. Virology. 278 (2): 301-8, each of which is incorporated herein by reference in its entirety.

The present disclosure provides an rAAV viral vector comprising: a) any of the rAAV vectors described herein; and b) an AAV capsid protein.

An AAV capsid protein can be any AAV capsid protein known in the art. In some aspects, the AAV capsid protein is a modified AAV capsid protein. An AAV capsid protein can be an AAV1 capsid protein, an AAV2 capsid protein, an AAV4 capsid protein, an AAV5 capsid protein, an AAV6 capsid protein, an AAV7 capsid protein, an AAV8 capsid protein, an AAV9 capsid protein, an AAV10 capsid protein, an AAV11 capsid protein, an AAV12 capsid protein, an AAV13 capsid protein, an AAVPHP.B capsid protein, an AAVrh74 capsid protein or an AAVrh.10 capsid protein. An AAV capsid protein can be any modified AAV capsid protein of the disclosure.

### Modified AAV Capsid Proteins

As used herein, the term “viral capsid” or “capsid” refers to the proteinaceous shell or coat of a viral particle. Capsids function to encapsidate, protect, transport, and release into the host cell a viral genome. Capsids are generally comprised of oligomeric structural subunits of protein (“capsid proteins”). As used herein, the term “encapsidated” means enclosed within a viral capsid. Provided herein are modified AAV capsid proteins which may be used to construct modified and/or chimeric AAV vectors or AAV capsids.

An AAV capsid generally consists of a total of 60 molecules of viral proteins (VPs), VP1, VP2, and VP3 at a ratio of about 1:1:10. VP1, VP2, and VP3 are encoded by the cap open reading frame and are generated through alternative splicing of the mRNA and use of an alternate translational start codon. The VP3 sequence of about 524-544 amino acids (aa) is shared among all VPs, the VP2 sequence is approximately 57aa longer than VP3 (about 580-601aa) and the VP1 sequence is approximately 137 aa longer than VP2 (about 713-738aa). The VP3 common region assembles the icosahedral capsid. See Wörner et al. Nature Communications Vol. 12, Article number: 1642 (2021).

In some aspects, modified AAV capsid proteins of the disclosure are derived from any AAV serotype known in the art. An AAV capsid protein can be derived from any AAV capsid protein known in the art. In some aspects, an AAV capsid protein can be derived from an AAV1 capsid protein, an AAV2 capsid protein, an AAV4 capsid protein, an AAV5 capsid protein, an AAV6 capsid protein, an AAV7 capsid protein, an AAV8 capsid protein, an AAV9 capsid protein, an AAV10 capsid protein, an AAV11 capsid protein, an AAV12 capsid protein, an AAV13 capsid protein, an AAVPHP.B capsid protein, an AAVrh8 capsid protein, an AAVrh74 capsid protein or, AAV-TT (AAVv66) capsid protein, an AAV PO1 capsid protein, an AAVDJ, or an AAVrh10 capsid protein. In some aspects, the AAV capsid protein is an AAV-TT capsid protein. In some aspects, the AAV capsid protein is an AAVrh10 capsid protein. In some aspects, modified AAV capsid proteins of the disclosure can be chimeric AAV capsid proteins derived from two or more AAV capsid proteins.

Disclosed herein are modified AAV capsid protein sequences. As used herein a “modified AAV capsid protein” or a “modified capsid protein” refers to AAV capsid proteins that have been modified with respect to the wild-type AAV capsid protein sequence. Modified AAV capsid proteins can comprise any one of capsid proteins VP1, VP2, or VP3. Modifications to AAV capsid protein sequences can be any protein modification known in the art including amino acid deletions, mutations, insertions, or re-arrangements. Modifications to AAV capsid proteins can be the formation of chimeric AAV capsid proteins wherein regions of two or more AAV capsid proteins are spliced or combined together to form a hybrid or chimeric AAV capsid protein. In some aspects, a modified AAV capsid protein comprised of a hybrid or chimeric AAV capsid protein comprises regions of two or more AAV capsid proteins each having a unique serotype. In some aspects, a hybrid capsid protein is one where variable domain loop regions of the capsid protein has been swapped. In some aspects, a hybrid AAV capsid protein comprises variable region loops from two or more capsid sequences having different serotypes.

Modified AAV capsid proteins of the disclosure can comprise insertions of peptides from any protein or peptide known in the art. In some aspects, the inserted peptide can be derived from a non-AAV capsid protein.

Modified AAV capsid proteins of the disclosure can comprise any combination of modifications. By way of non-limiting example, AAV capsid proteins of the disclosure can be both chimeric and contain at least one of an amino acid deletion, mutation, insertion, or re-arrangement.

Modified AAV capsid proteins of the disclosure can used to form AAV capsids with improved properties including increased transduction in a specific tissue type (i.e. “on-target transduction” and/or reduced transduction in undesired tissue types (i.e. “off-target transduction”). In some aspects, muscle tissue-specific transduction is observed. In some aspects, ocular tissue specific transduction is observed. In some aspects, neuron or neuronal tissue specific transduction is observed. In some aspects, transduction in the liver is reduced or eliminated. In some aspects, AAV capsids comprising modified AAV capsid proteins have reduced transduction in non-targeted tissues or cell types. In some aspects, non-targeted tissues include liver, lung, kidney, brain, spleen, intestine, spinal cord, or reproductive organs.

Without wishing to be bound by theory, AAV vectors comprising modified AAV capsid proteins of the disclosure can have reduced reactivity to pre-existing neutralizing antibodies in a human subject due to the modifications to the capsid producing distinct binding epitopes not observed in commonly used AAV capsid serotypes.

### Peptide Insertions in AAV Capsid Proteins

Peptides or amino acids can be inserted in any region of an AAV capsid protein. Insertions can occur at the N-terminus or C-terminus of the protein. In some aspects, insertions can occur in any variable region (VR) of the capsid protein including VR1 (VRI), VR2 (VRII), VR3 (VRIII), VR4 (VRIV), VR5 (VRV), VR6 (VRVI, VR7 (VRVII), VR8 (VRVIII), or VR9 (VRX).

In some aspects, modified AAV capsid proteins of the disclosure comprise modified variable regions. In some aspects, modified AAV capsid proteins of the disclosure comprise modified VR VIII regions. In some aspects, the modification to the AAV capsid protein is an insertion into VR VIII.

### Peptide Insertion Sequences

Peptides sequences were chosen to enhance transduction in a tissue-specific manner. Any sequence that enhances tissue-specific transduction is contemplated. In some aspects, muscle tissue-specific transduction is observed. In some aspects, ocular tissue specific transduction is observed. In some aspects, neuron or neuronal tissue specific transduction is observed. Inserted peptides can target tissue-specific receptors leading to increased transduction in said tissues.

In some aspects, modified AAV capsid proteins comprise a peptide insertion targeting insulin receptor (INSR). AAV vectors designed to target INSR have been shown to enhance intramuscular transduction (See Jackson et al. Molecular Therapy Methods & Clinical Development, 2020, 19, 11, 496-506 which is incorporated herein by reference in its entirety). In some aspects, the inserted INSR-targeting peptide is an insulin-mimetic peptide referred to as S519. In some aspects, the S519 peptide can comprise, consist essentially of, or consist of an amino acid sequence at least 65%, 70%, 75%, 80%, 85%, 90%, 95%, 96%, 97%, 98%, 99% or 100% (or any percentage in between) identical to the amino acid sequence SLEEEWAQVECEVYGRGCPSGSLDESFYDWFERQL (SEQ ID NO: 301).

In some aspects, modified AAV capsid proteins comprise a peptide insertion targeting muscle-specific kinase (MUSK). MUSK expression in the liver has been shown to be either extremely low or absent. Acetylcholinesterase collagenic tail peptide (ColQ) is known to bind and target MUSK. In some aspects, the inserted MUSK-targeting peptide is a C-terminal portion of ColQ (ColQ CTD). In some aspects, the ColQ CTD peptide can comprise, consist essentially of, or consist of an amino acid sequence at least 65%, 70%, 75%, 80%, 85%, 90%, 95%, 96%, 97%, 98%, 99% or 100% (or any percentage in between) identical to the amino acid sequence TPFYPVGYTVKQPGTCGDGVLQPGEECDDGNPDVSDGCIDCHRAYCGDGYRHQGV EDCDGSDFGYLTCETYLPGSYGDLRCTQYCSIDSTPCRYFT (SEQ ID NO: 302).

In some aspects, modified AAV capsid proteins comprise a peptide insertion targeting integrin. In some aspects, the integrin-targeting peptide comprises an RGD-motif. RGD sequences are known in the art, and include, for example, the motif RGDXXXX which may be inserted in an AAV viral vector for targeting via the integrin class of receptors. see, e.g., Michelfelder et al., PLoS One. 2009; 4(4): e5122 which is incorporated herein by reference in its entirety for example of RGD sequences that may be used in modified AAV capsids described herein. RGD-motif peptide insertions into VR8 of AAV9 has been shown to increase mouse muscle transduction (See Weinmann et al. Nature Communications, 11:5432 which is incorporated herein by reference in its entirety). In some aspects, the RGD peptide comprises a subsequence Y or F amino acid to produce an RGDY or RGDF motif. RGDY or RGDF motifs have been demonstrated to produce enhanced muscle transduction in non-human primates (NHP) (See Tabebordbar et al. Cell, 184, 19, 2021, 4919-4938 which is incorporated herein by reference in its entirety). In some aspects, the RGD sequence comprises RGDLGLS (SEQ ID NO: 303). In some aspects, the RGD sequence comprises RGDLSTP (SEQ ID NO: 304), SNSRGDYNSL (SEQ ID NO: 305), ENRRGDFNNT (SEQ ID NO: 306), SRGDYNSL (SEQ ID NO: 307), RGDYNSL (SEQ ID NO: 308), RGDLST (SEQ ID NO: 309), or RGDYVGL (SEQ ID NO: 310).

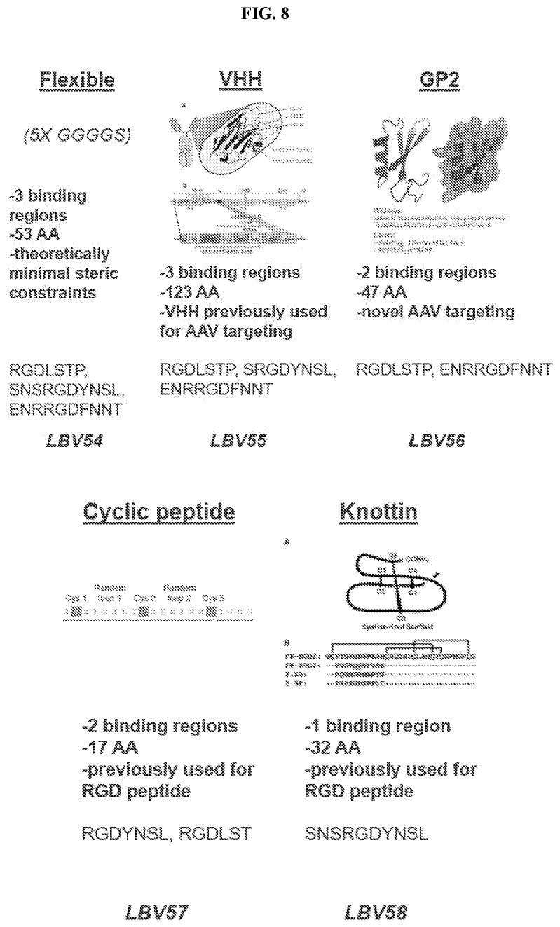

In some aspects, RGD sequences of the disclosure can be inserted in a scaffold (FIG. 8). In some aspects, RGD sequences of the disclosure comprise a linker on one or more of the N-terminus and C-terminus forming a linker scaffold. In some aspects, the linker scaffold comprises a flexible linker such as GGGS (SEQ ID NO: 311). In some aspects, the linker scaffold comprises a rigid scaffold such as a VHH, GP2, cyclic peptide, or knottin scaffold.

In some aspects, the RGD sequence comprises a variable domain of camelid heavy-chain-only antibody (VHH) RGD peptide, for example a VHH RGD peptide comprising, consisting essentially of, or consisting of an amino acid sequence at least 65%, 70%, 75%, 80%, 85%, 90%, 95%, 96%, 97%, 98%, 99% or 100% (or any percentage in between) identical to the amino acid sequence EVQLQASGGGFVQAGGSLRLSCAVSGRGDLSTPSYGMHWVRQAPGKEREFVAGISR GDYNSLYYADSVQGRFTISRDNAKNTVYLQMNSLKPEDTATYYCAENRRGDFNNT YWGQGTQVTVSS (SEQ ID NO:316) (RGD motif underlined).

In some aspects, RGD peptide insertions comprise a glycoprotein 2 (GP2) peptide scaffolded insertion. In some aspects, GP2 scaffolded peptide insertions comprise the amino acid sequence KFWATVGRGDLSTPFEVPVYAETLDEALELAENRRGDFNNTVTRVRP (SEQ ID NO:317) or GGGGSGGGGSKFWATVGRGDLSTPFEVPVYAETLDEALELAENRRGDFNNTVTRVRPGGGG S (SEQ ID NO: 318).

In some aspects, RGD peptide insertions can comprise a knottin scaffolded peptide insertion. In some aspects, knottin scaffolded peptide insertions comprise the amino acid sequence NSRGDYNSLSCSQDSDCLAGCVCGPNGFC (SEQ ID NO: 319) or GGGGSGGGGSGCSNSRGDYNSLSCSQDSDCLAGCVCGPNGFCGGGGGS (SEQ ID NO: 320).

In some aspects, RGD peptide insertions can comprise a cyclic peptide scaffolded RGD peptide insertion. In some aspects, cyclic peptide scaffolded RGD peptide insertions comprise the amino acid sequence ACRGDYNSLCRGDLSTC (SEQ ID NO: 321) or GGGGACRGDYNSLCRGDLSTCGGGGS (SEQ ID NO: 322).

Scaffolds can be used to insert any peptide known in the art. Therefore, VHH, GP2, cyclic peptides, knottin, or flexible linkers can be used to scaffold or flank any peptide for insertion into a modified AAV capsid protein of the disclosure.

Inserted peptides can be flanked on the N-terminus or C-terminus by flexible linker peptides of any length. In some aspects, a flexible linker such as GGGS (SEQ ID NO: 311) is used. In some aspects, the flexible GGGS linker can be repeated multiple times to form a longer linker sequence. In some aspects, the linker is repeated one time, two times, three times, four times, five times, six times, seven times, eight times, nine times, or ten times. In some aspects, the linker sequence comprises GGGGS (SEQ ID NO: 311), GGGGSGGGGS (SEQ ID NO: 312); GGGGSGGGGSGGGGS(SEQ ID NO: 313); GGGGSGGGGSGGGGSGGGGS (SEQ ID NO: 314); or GGGGSGGGGSGGGGSGGGGSGGGGS (SEQ ID NO: 315).

### Point Mutations

Additionally or alternatively, modified AAV capsid proteins may comprise amino acid mutations yielding increased transduction in a desired tissue type. In some aspects, modified AAV capsid proteins may comprise amino acid mutations yielding reduced transduction in specific tissues types. In some aspects, modified AAV capsid proteins comprise amino acid mutations that yield reduced liver tissue transduction.

In some embodiments, a modified AAV capsid protein comprises an amino acid sequence provided herein (e.g., a sequence selected from Table 1) with one, two, three, four, five, six, seven, eight, nine, ten, or more amino acid changes. The amino acid change can be the substitution of one amino acid for any other amino acid, including natural amino acids and un-natural or modified amino acids. In some aspects, the mutation is a conservative amino acid mutation. A conservative amino acid substitution is an amino acid replacement in a protein that changes a given amino acid to a different amino acid with similar biochemical properties (e.g., charge, hydrophobicity or size). Examples of conservative amino acid substitutions include the substitution of one hydrophobic residue such as isoleucine, valine, leucine or methionine for another; or the substitution of one charged or polar residue for another, such as the substitution of arginine for lysine, glutamic acid for aspartic acid, glutamine for asparagine, and the like. In some embodiments, a conservative amino acid substitution is selected from alanine to serine; asparagine to glutamine or histidine; aspartate to glutamate; cysteine to serine; glycine to proline; histidine to asparagine or glutamine; lysine to arginine, glutamine, or glutamate; phenylalanine to tyrosine, serine to threonine; threonine to serine; tryptophan to tyrosine; tyrosine to tryptophan or phenylalanine.

In some aspects, mutations at position 503 of AAVrh8 yield reduced liver transduction. In some aspects, substitution of tryptophan for alanine at position 503 of AAVrh8 yields reduced liver transduction. In some aspects, mutations at position 505 of AAVrh74 yields reduced liver transduction. In some aspects, substitution of tryptophan for alanine at position 55 of AAVrh74 yields reduced liver transduction. In some aspects, mutations at position 498 of an AAV9 capsid proteins yield reduced liver transduction. In some aspects, an AAV9 point mutation comprises an N498 mutation. In some aspects, an AAV9 point mutation comprises an N498I mutation. In some aspects, mutations at position 602 of an AAV capsid proteins yield reduced liver transduction.

Robust liver de-targeting in NHPs has been suggested by mutating the following residues in AAV9 (F501I, G505R, Y706C). In some aspects, liver de-targeting mutations of AAV9 include at least one of F501I, G505R, Y706C. Equivalent mutations in certain vectors of the disclosure include F503I, G507R, and Y708C in LBV30 to generate LBV92. In some aspects, AAV Rh74 liver de-targeting mutations comprise mutations to any combination of residues F503, G507, Y707, and/or Y708 of AAV Rh74. On some aspects, these mutations comprise F503I, G507R, Y707C, and/or Y708C of AAV Rh74.

In some aspects, point mutations of the disclosure are numbered in reference to the wild-type AAV capsid protein sequence. Wild type AAV capsid proteins sequences are disclosed in table XXX. In some aspects, modified AAV capsid proteins of the disclosure comprise insertions and/or deletions or variable region swaps that may alter the length of capsid protein sequence. It should be understood that point mutations referenced in table X1 are in reference to the wild type or unmodified AAV capsid protein sequence.

### Modified AAV Capsid Sequences

The disclosure provides modified AAV capsid protein amino acid sequences. The disclosure further provides nucleic acid sequences that encode modified AAV capsid proteins of the disclosure.

Modified AAV capsid proteins of the disclosure are set forth in Table 1. Table 1 lists the ID of the assembled AAV capsid comprising VP1, VP2, and VP3 capsid proteins including modified AAV capsid proteins as disclosed herein. Table 1 describes the serotype of the capsid protein as well as any modifications made to the capsid sequence.

TABLE 1
AAV capsid protein sequences of the disclosure

| | | | | VP1 | VP2/VP3 | VP1 | VP2/VP3 |
|---|---|---|---|---|---|---|---|
| | | | | amino | amino | nucleic | nucleic |
| | | | | acid | acid | acid | acid |
| | | | | SEQ | SEQ | SEQ | SEQ |
| Viral ID | VP1 | VP2/VP3 | Application | ID NO: | ID NO: | ID NO: | ID NO: |
| | Rh74 RGD | | | 10 | 10 | 27 | 27 |
| | insertion | | | | | | |
| | Rh8 RGD | | | 11 | 11 | 28 | 28 |
| | insertion | | | | | | |
| | Rh74 S371-VP2 | Rh74 S371-VP2 | | 12 | 12 | 29 | 29 |
| | (VP1 and VP3 | (VP1 and VP3 | | | | | |
| | wildtype Rh74) | wildtype Rh74) | | | | | |
| | Rh74 S519-VP2 | Rh74 S519-VP2 | | 13 | 13 | 30 | 30 |
| | (VP1 and VP3 | (VP1 and VP3 | | | | | |
| | wildtype Rh74) | wildtype Rh74) | | | | | |
| | Rh74 S961-VP2 | Rh74 S961-VP2 | | 14 | 14 | 31 | 31 |
| | (VP1 and VP3 | (VP1 and VP3 | | | | | |
| | wildtype Rh74) | wildtype Rh74) | | | | | |
| | Rh74 VP1 S519 | Rh74 VP1 S519 | | 15 | 15 | 32 | 32 |
| | (VP2 and VP3 | (VP2 and VP3 | | | | | |
| | wildtype Rh74) | wildtype Rh74) | | | | | |
| | Rh74 VP1 S961 | Rh74 VP1 S961 | | 16 | 16 | 33 | 33 |
| | (VP2 and VP3 | (VP2 and VP3 | | | | | |
| | wildtype Rh74) | wildtype Rh74) | | | | | |
| | Rh74 VP1 MuSK | Rh74 VP1 MuSK | | 17 | 17 | 34 | 34 |
| | VHH (VP2 and VP3 | VHH (VP2 and VP3 | | | | | |
| | wildtype Rh74) | wildtype Rh74) | | | | | |
| | AAVDJ 7m8 (AA) | | In vitro | 231 | 231 | 232 | 232 |
| | | | transduction | | | | |
| LBV9 | Rh8 W503A | | IV, muscle | 2 | 2 | 19 | 19 |
| LBV10 | Rh74 W505A | | IV, muscle | 1 | 1 | 18 | 18 |
| LBV11 | AAV9 VP1, 2 Rh8 | | IV, muscle | 3 | 3 | 20 | 20 |
| | VP3 | | | | | | |
| LBV12 | AAV8 VP1, 2 Rh8 | | IV, muscle | 4 | 4 | 21 | 21 |
| | VP3 | | | | | | |
| LBV13 | AAV1 VP1, 2 Rh8 | | IV, muscle | 5 | 5 | 22 | 22 |
| | VP3 | | | | | | |
| LBV14 | AAV7 VP1, 2 Rh8 | | IV, muscle | 6 | 6 | 23 | 23 |
| | VP3 | | | | | | |
| LBV15 | AAV9 VP1, 2 Rh74 | | IV, muscle | 7 | 7 | 24 | 24 |
| | VP3 | | | | | | |
| LBV16 | AAV1 VP1, 2 Rh74 | | IV, muscle | 8 | 8 | 25 | 25 |
| | VP3 | | | | | | |
| LBV17 | AAV7 VP1, 2 Rh74 | | IV, muscle | 9 | 9 | 26 | 26 |
| | VP3 | | | | | | |
| LBV18 | Rh8 VR-VIII P1 | | IV, muscle | — | — | — | — |
| LBV19 | Rh74 VR-VIII P1 | | IV, muscle | — | — | — | — |
| LBV20 | AAV9 with VR-IV | | IV, muscle | — | — | — | — |
| | and HI loop from | | | | | | |
| | Rh8 | | | | | | |
| LBV21 | AAV9 with VR-VIII | | IV, muscle | — | — | — | — |
| | and HI loop from | | | | | | |
| | Rh8 | | | | | | |
| LBV22 | AAV9 with VR-IV | | IV, muscle | — | — | — | — |
| | and HI loop from | | | | | | |
| | Rh74 | | | | | | |
| LBV23 | AAV9 with VR-VIII | | IV, muscle | — | — | — | — |
| | and HI loop from | | | | | | |
| | Rh74 | | | | | | |
| LBV24 | AAV8 with VR-IV | | IV, muscle | — | — | — | — |
| | from Rh8 | | | | | | |
| LBV25 | AAV8 with VR-VIII | | IV, muscle | — | — | — | — |
| | from Rh8 | | | | | | |
| LBV26 | AAV8 with VR-IV | | IV, muscle | — | — | — | — |
| | from Rh74 | | | | | | |
| LBV27 | AAV8 with VR-VIII | | IV, muscle | — | — | — | — |
| | from Rh74 | | | | | | |
| LBV29 | Rh74 VR8 mColQ | Rh74 (C01687) | IV, muscle | 35 | 84 | 133 | 181 |
| | (C01692) | | | | | | |
| LBV30 | Rh74 VR8 mColQ | Rh74 VR8 P1 | IV, muscle | 36 | 85 | 134 | 182 |
| | (C01692) | (C01765) | | | | | |
| LBV31 | PO1 VR8 mColQ | PO1 VR8 P1 | IV, muscle | 37 | 86 | 135 | 183 |
| | (C01839) | (C01840) | | | | | |
| LBV32 | Rh74 VR8 mColQ | PO1 VR8 P1 | IV, muscle | 38 | 87 | 136 | 184 |
| | (C01692) | (C01840) | | | | | |
| LBV33 | PO1 BR2mut VR8 | PO1 VR8 BR2mut | IV, muscle | 39 | 88 | 137 | 185 |
| | mColQ (C02036) | P1 (C02037) | | | | | |
| LBV34 | Rh74/PO1 VR8 | PO1 VR8 P1 | IV, muscle | 40 | 89 | 138 | 186 |
| | mColQ (C02038) | (C01840) | | | | | |
| LBV35 | AAV4/PO1 VR8 | PO1 VR8 P1 | IV, muscle | 41 | 90 | 139 | 187 |
| | mColQ (C02039) | (C01840) | | | | | |
| LBV41 | Rh74 VR8 mColQ | AAV9 VR8 P1 | IV, muscle | 42 | 91 | 140 | 188 |
| | (C01692) | (C01837) | | | | | |
| LBV42 | Rh74 VR8 P1 | AAV9 VR8 P1 | IV, muscle | 43 | 92 | 141 | 189 |
| | (C02041) | (C01837) | | | | | |
| LBV43 | Rh74 VR8 P1 | PO1 VR8 P1 | IV, muscle | 44 | 93 | 142 | 190 |
| | (C02041) | (C01840) | | | | | |
| LBV44 | AAV4/Rh74 VR8 | Rh74 VR8 P1 | IV, muscle | 45 | 94 | 143 | 191 |
| | ColQ (C02076) | (C01765) | | | | | |
| LBV45 | AAV4/Rh74 VR8 | Rh74 VR8 P1 | IV, muscle | 46 | 95 | 144 | 192 |
| | P1 (C02077) | (C01765) | | | | | |
| LBV46 | AAV9 VR8 ColQ | Rh74 VR8 P1 | IV, muscle | 47 | 96 | 145 | 193 |
| | (C01836) | (C01765) | | | | | |
| LBV47 | AAV9 VR8 P1 | Rh74 VR8 P1 | IV, muscle | 48 | 97 | 146 | 194 |
| | (C02086) | (C01765) | | | | | |
| LBV48 | AAV9 VR8 P1 | PO1 VR8 P1 | IV, muscle | 49 | 98 | 147 | 195 |
| | (C02086) | (C01840) | | | | | |
| LBV49 | PO1 VR8 mColQ | PO1 VR8 P1mod | IV, muscle | 50 | 99 | 148 | 196 |
| | (C01839) | (C02095) | | | | | |
| LBV50 | PO1 BR2mut VR8 | PO1 BR2mut VR8 | IV, muscle | 51 | 100 | 149 | 197 |
| | mColQ (C02036) | P1mod (C02096) | | | | | |
| LBV54 | AAV9 VR8 GGGGS | AAV9 | IV, muscle | 52 | 101 | 150 | 198 |
| | RGD | | | | | | |
| LBV55 | AAV9 VR8 VHH | AAV9 | IV, muscle | 53 | 102 | 151 | 199 |
| | RGD | | | | | | |
| LBV56 | AAV9 VR8 GP2 | AAV9 | IV, muscle | 54 | 103 | 152 | 200 |
| | RGD | | | | | | |
| LBV57 | AAV9 VR8 cRGD | AAV9 | IV, muscle | 55 | 104 | 153 | 201 |
| LBV58 | AAV9 VR8 Knottin | AAV9 | IV, muscle | 56 | 105 | 154 | 202 |
| | RGD | | | | | | |
| LBV59 | AAVpo1 VR8 | AAVpo1 | IV, muscle | 57 | 106 | 155 | 203 |
| | GGGGS RGD | | | | | | |
| LBV60 | AAVpo1 VR8 VHH | AAVpo1 | IV, muscle | 58 | 107 | 156 | 204 |
| | RGD | | | | | | |
| LBV61 | AAVpo1 VR8 GP2 | AAVpo1 | IV, muscle | 59 | 108 | 157 | 205 |
| | RGD | | | | | | |
| LBV62 | AAVpo1 VR8 | AAVpo1 | IV, muscle | 60 | 109 | 158 | 206 |
| | cRGD | | | | | | |
| LBV63 | AAVpo1 VR8 | AAVpo1 | IV, muscle | 61 | 110 | 159 | 207 |
| | Knottin RGD | | | | | | |
| LBV64 | AAV9 VR8 GGGGS | AAV9 VR8 | IV, muscle | 62 | 111 | 160 | 208 |
| | RGD | RGDLSTP | | | | | |
| LBV65 | AAV9 VR8 VHH | AAV9 VR8 | IV, muscle | 63 | 112 | 161 | 209 |
| | RGD | RGDLSTP | | | | | |
| LBV66 | AAV9 VR8 GP2 | AAV9 VR8 | IV, muscle | 64 | 113 | 162 | 210 |
| | RGD | RGDLSTP | | | | | |
| LBV67 | AAV9 VR8 cRGD | AAV9 VR8 | IV, muscle | 65 | 114 | 163 | 211 |
| | | RGDLSTP | | | | | |
| LBV68 | AAV9 VR8 Knottin | AAV9 VR8 | IV, muscle | 66 | 115 | 164 | 212 |
| | RGD | RGDLSTP | | | | | |
| LBV69 | AAVpo1 VR8 | AAVpo1 VR8 | IV, muscle | 67 | 116 | 165 | 213 |
| | GGGGS RGD | RGDLSTP | | | | | |
| LBV70 | AAVpo1 VR8 VHH | AAVpo1 VR8 | IV, muscle | 68 | 117 | 166 | 214 |
| | RGD | RGDLSTP | | | | | |
| LBV71 | AAVpo1 VR8 GP2 | AAVpo1 VR8 | IV, muscle | 69 | 118 | 167 | 215 |
| | RGD | RGDLSTP | | | | | |
| LBV72 | AAVpo1 VR8 | AAVpo1 VR8 | IV, muscle | 70 | 119 | 168 | 216 |
| | cRGD | RGDLSTP | | | | | |
| LBV73 | AAVpo1 VR8 | AAVpo1 VR8 | IV, muscle | 71 | 120 | 169 | 217 |
| | Knottin RGD | RGDLSTP | | | | | |
| LBV76 | PO1 VR8 mColQ | AAVpo1 VR8 | IV, muscle | 72 | 121 | 170 | 218 |
| | (C01839) | RGDLSTP | | | | | |
| LBV91 | AAVHSC16 VR8 | AAVHSC16 | IV, muscle | 73 | 122 | 171 | 219 |
| | VHH RGD | | | | | | |
| LBV92 | Rh74 VR8 mColQ | Rh74 VR8 P1 F503I | IV, muscle | 74 | 123 | 172 | 220 |
| | F503I G507R | G507R Y707C | | | | | |
| | Y707C | | | | | | |
| LBV93 | Rh74 VR8 mColQ | Rh74 VR8 | IV, muscle | 75 | 124 | 173 | 221 |
| | F503I G507R | RDGYVGL F503I | | | | | |
| | Y707C | G507R Y707C | | | | | |
| LBV94 | Rh74 VR8 4A | Rh74 VR8 | IV, muscle | 76 | 125 | 174 | 222 |
| | F503I G507R | RGDYVGL F503I | | | | | |
| | Y707C | G507R Y707C | | | | | |
| LBV95 | Rh74 VR8 4x z8 | Rh74 VR8 | IV, muscle | 77 | 126 | 175 | 223 |
| | F503I G507R | RGDYVGL F503I | | | | | |
| | Y707C | G507R Y707C | | | | | |
| LBV96 | AAVHSC16 VR8 | AAVHSC16 | IV, muscle | 78 | 127 | 176 | 224 |
| | Slit2 LG | | | | | | |
| LBV97 | AAVHSC16 VR8 | AAVHSC16 VR8 | IV, muscle | 79 | 128 | 177 | 225 |
| | Slit2 LG | RGDYVGL | | | | | |
| LBV98 | AAV9 VR8 Slit2 LG | AAV9 VR8 | IV, muscle | 80 | 129 | 178 | 226 |
| | | RGDYVGL | | | | | |
| LBV99 | Rh74 VR8 Slit2 LG | Rh74 F503I G507R | IV, muscle | 81 | 130 | 179 | 227 |
| | F503I G507R | Y707C | | | | | |
| | Y707C | | | | | | |
| LBV100 | Rh74 VR8 Slit2 LG | Rh74 VR8 | IV, muscle | 82 | 131 | 180 | 228 |
| | F503I G507R | RGDYVGL F503I | | | | | |
| | Y707C | G507R Y707C | | | | | |
| LBV101 | AAV9 VR4 swap | AAV9 VR4 swap | IV, muscle | 83 | — | — | 229 |
| | DGAATKN VR8 | DGAATKN VR8 | | | | | |
| | RGDYVGL | RGDYVGL | | | | | |
| LBV102 | AAV9 VR4 swap | AAV9 VR4 swap | IV, muscle | — | 132 | — | 230 |
| | TTGGHSS VR8 | TTGGHSS VR8 | | | | | |
| | RGDYVGL | RGDYVGL | | | | | |
| LBV28 | Rh74 VP1-S519 | Rh74 (C01687) | In vitro | 233 | 236 | 238 | 241 |
| | (C01688) | | transduction | | | | |
| LBV40 | Rh74 VP1 VR8 | Rh74 (C01687) | in vitro, lung, | 234 | 237 | 239 | 242 |
| | FnIII MSLN | | tumor | | | | |
| | (C02040) | | | | | | |
| LBV77 | AAVDJ 7m8 | AAVDJ 7m8 | In vitro | 235 | 235 | 240 | 240 |
| | (C01442) | (C01442) | transduction | | | | |
| LBV108 | Rh74 VR8 NK1 | Rh74 | Liver (hepatocyte) | 243 | 256 | 268 | 281 |
| | K1D | | targeting | | | | |
| LBV109 | Rh74 VR8 NK1 LE | Rh74 | Liver (hepatocyte) | 244 | 257 | 269 | 282 |
| | | | targeting | | | | |
| LBV110 | Rh74 VR8 VHH | Rh74 VR8 | Muscle-targeting, | 245 | 258 | 270 | 283 |
| | RGD F503I G507R | RGDYVGL F503I | IV delivery | | | | |
| | Y708C | G507R Y708C | | | | | |
| LBV111 | Rh74 VR8 VHH | Rh74 F503I G507R | Muscle-targeting, | 246 | 259 | 271 | 284 |
| | RGD F503I G507R | Y708C | IV delivery | | | | |
| | Y708C | | | | | | |
| LBV112 | AAV9 VR8 VHH | AAV9 Q590A | Muscle-targeting, | 247 | 260 | 272 | 285 |
| | RGD | | IV delivery | | | | |
| LBV113 | AAV9 VR8 VHH | AAV9 N498I | Muscle-targeting, | 248 | 261 | 273 | 286 |
| | RGD N498I | | IV delivery | | | | |
| LBV114 | AAV9 VR8 VHH | AAV9 VR8 | Muscle-targeting, | 249 | 262 | 274 | 287 |
| | RGD N498I | RGDYVGL N498I | IV delivery | | | | |
| LBV115 | AAV9 VR8 VHH | AAV9 586 QQNAA | Muscle-targeting, | 250 | 263 | 275 | 288 |
| | RGD | 590 | IV delivery | | | | |
| LBV116 | AAV9 VR8 VHH | AAV9 VR4 swap | Muscle-targeting, | 251 | 264 | 276 | 289 |
| | RGD | TTGGHSS VR8 | IV delivery | | | | |
| | | RGDYVGL | | | | | |
| LBV117 | AAV9 VR8 VHH | AAV9 G505R | Muscle-targeting, | 252 | 265 | 277 | 290 |
| | RGD G505R | | IV delivery | | | | |
| LBV118 | AAV9 VR8 VHH | AAV9 VR8 | Muscle-targeting, | 253 | 266 | 278 | 291 |
| | RGD G505R | RGDYVGL G505R | IV delivery | | | | |
| LBV120 | Rh74 F503I | Rh74 F503I G507R | Muscle-targeting, | 254 | 254 | 279 | 279 |
| | G507R Y708C | Y708C | IV delivery | | | | |
| LBV121 | AAV9 VR8 VHH | AAV9 VR8 | Muscle-targeting, | 255 | 267 | 280 | 292 |
| | RGD | RGDYVGL | IV delivery | | | | |
| | Wild-type AAV | | | 293 | 293 | 297 | 297 |
| | Rh74 | | | | | | |
| | Wild-type AAV2 | | | 294 | 294 | 298 | 298 |
| | Wild-type AAV9 | | | 295 | 295 | 299 | 299 |
| | Wild-type AAV | | | 296 | 296 | 300 | 300 |
| | Rh10 | | | | | | |

In some aspects, a modified AAV capsid VP1 protein provided herein comprises, consists essentially of, or consists of an amino acid sequence that is at least 80%, at least 85%, at least 90%, at least 95%, at least 96%, at least 97%, at least 98%, or at least 99%, or at least 100% (or any percentage in between) identical to a VP1 sequence set forth in Table 1.

In some embodiments, a modified AAV capsid VP2/3 protein provided herein comprises an amino acid sequence that is at least 80%, at least 85%, at least 90%, at least 95%, at least 96%, at least 97%, at least 98%, at least 99%, or at least 100% (or any percentage in between) identical to a VP2/VP3 sequence set forth in Table 1.

In some embodiments, a modified AAV capsid VP1 protein provided herein is encoded by a polynucleotide comprising a sequence that is at least 80%, at least 85%, at least 90%, at least 95%, at least 96%, at least 97%, at least 98%, or at least 99%, or at least 100% (or any percentage in between) identical to the VP1 sequence set forth in Table 1.

In some embodiments, a modified AAV capsid VP2/3 protein provided herein is encoded by a polynucleotide comprising a sequence that is at least 80%, at least 85%, at least 90%, at least 95%, at least 96%, at least 97%, at least 98%, or at least 99%, or at least 100% (or any percentage in between) identical to the VP2/VP3 sequence set forth in Table 1.

In some aspects, a muscle-targeted modified AAV capsid VP1 protein provided herein comprises, consists essentially of, or consists of an amino acid sequence that is at least 80%, at least 85%, at least 90%, at least 95%, at least 96%, at least 97%, at least 98%, or at least 99%, or at least 100% (or any percentage in between) identical to a VP1 sequence set forth in any Table 1.

In some embodiments, a muscle-targeted modified AAV capsid VP2/3 protein provided herein comprises an amino acid sequence that is at least 80%, at least 85%, at least 90%, at least 95%, at least 96%, at least 97%, at least 98%, at least 99%, or at least 100% (or any percentage in between) identical to a VP2/VP3 sequence set forth in Table 1.

In some embodiments, a muscle-targeted modified AAV capsid VP1 protein provided herein is encoded by a polynucleotide comprising a sequence that is at least 80%, at least 85%, at least 90%, at least 95%, at least 96%, at least 97%, at least 98%, or at least 99%, or at least 100% (or any percentage in between) identical to a VP1 sequence set forth in Table 1.

In some embodiments, a muscle-targeted modified AAV capsid VP2/3 protein provided herein is encoded by a polynucleotide comprising a sequence that is at least 80%, at least 85%, at least 90%, at least 95%, at least 96%, at least 97%, at least 98%, or at least 99%, or at least 100% (or any percentage in between) identical to a VP2/VP3 sequence set forth in Table 1.

In another aspect, provided herein are modified AAV capsid proteins that are useful for in vitro transduction.

In some aspects, a modified VP1 capsid protein sequence is at least 80%, at least 85%, at least 90%, at least 95%, at least 96%, at least 97%, at least 98%, or at least 99%, or at least 100% (or any percentage in between) identical to SEQ ID NO: 53. In some aspects, SEQ ID NO: 53 is an AAV9 VP1 capsid protein comprising a VHH RGD peptide insertion. In some aspects, the modified VP1 capsid protein set forth in SEQ ID NO: 53 is encoded by a nucleic acid sequence at least 80%, at least 85%, at least 90%, at least 95%, at least 96%, at least 97%, at least 98%, or at least 99%, or at least 100% (or any percentage in between) identical to SEQ ID NO: 151.

In some aspects, a modified VP2/VP3 capsid protein sequence is at least 80%, at least 85%, at least 90%, at least 95%, at least 96%, at least 97%, at least 98%, or at least 99%, or at least 100% (or any percentage in between) identical to SEQ ID NO: 102. In some aspects, SEQ ID NO: 102 is an AAV9 VP2/3 capsid protein. In some aspects, the VP2/VP3 capsid protein set forth in SEQ ID NO: 102 is encoded by a nucleic acid sequence at least 80%, at least 85%, at least 90%, at least 95%, at least 96%, at least 97%, at least 98%, or at least 99%, or at least 100% (or any percentage in between) identical to SEQ ID NO: 199.

In some aspects, a modified VP1 capsid protein sequence is at least 80%, at least 85%, at least 90%, at least 95%, at least 96%, at least 97%, at least 98%, or at least 99%, or at least 100% (or any percentage in between) identical to SEQ ID NO: 75. In some aspects, SEQ ID NO: 75 is an Rh74 VP1 capsid protein comprising an mColQ peptide insertion and mutations F503I, G507R, and Y707C (amino acid numbering in reference to wild-type Rh74 capsid protein). In some aspects, the modified VP1 capsid protein set forth in SEQ ID NO: 75 is encoded by a nucleic acid sequence at least 80%, at least 85%, at least 90%, at least 95%, at least 96%, at least 97%, at least 98%, or at least 99%, or at least 100% (or any percentage in between) identical to SEQ ID NO: 173.

In some aspects, a modified VP2/VP3 capsid protein sequence is at least 80%, at least 85%, at least 90%, at least 95%, at least 96%, at least 97%, at least 98%, or at least 99%, or at least 100% (or any percentage in between) identical to SEQ ID NO: 124. In some aspects, SEQ ID NO: 124 is a modified Rh74 VP2/3 capsid protein comprising an RGD peptide insertion and mutations F503I, G507R, and Y707C (amino acid numbering in reference to wild-type Rh74 capsid protein). In some aspects, the VP2/VP3 capsid protein set forth in SEQ ID NO: 124 is encoded by a nucleic acid sequence at least 80%, at least 85%, at least 90%, at least 95%, at least 96%, at least 97%, at least 98%, or at least 99%, or at least 100% (or any percentage in between) identical to SEQ ID NO: 221.

In some aspects, a modified VP1 capsid protein sequence is at least 80%, at least 85%, at least 90%, at least 95%, at least 96%, at least 97%, at least 98%, or at least 99%, or at least 100% (or any percentage in between) identical to SEQ ID NO: 245. In some aspects, SEQ ID NO: 245 is an Rh74 VP1 capsid protein comprising VHH RGD peptide insertion and mutations F503I, G507R, and Y707C (amino acid numbering in reference to wild-type Rh74 capsid protein). In some aspects, the modified VP1 capsid protein set forth in SEQ ID NO: 245 is encoded by a nucleic acid sequence at least 80%, at least 85%, at least 90%, at least 95%, at least 96%, at least 97%, at least 98%, or at least 99%, or at least 100% (or any percentage in between) identical to SEQ ID NO: 270.

In some aspects, a modified VP2/VP3 capsid protein sequence is at least 80%, at least 85%, at least 90%, at least 95%, at least 96%, at least 97%, at least 98%, or at least 99%, or at least 100% (or any percentage in between) identical to SEQ ID NO: 258. In some aspects, SEQ ID NO: 258 is a modified Rh74 VP2/3 capsid protein comprising an RGD peptide insertion and mutations F503I, G507R, and Y707C (amino acid numbering in reference to wild-type Rh74 capsid protein). In some aspects, the VP2/VP3 capsid protein set forth in SEQ ID NO: 258 is encoded by a nucleic acid sequence at least 80%, at least 85%, at least 90%, at least 95%, at least 96%, at least 97%, at least 98%, or at least 99%, or at least 100% (or any percentage in between) identical to SEQ ID NO: 283.

In some aspects, a modified VP1 capsid protein sequence is at least 80%, at least 85%, at least 90%, at least 95%, at least 96%, at least 97%, at least 98%, or at least 99%, or at least 100% (or any percentage in between) identical to SEQ ID NO: 249. In some aspects, SEQ ID NO: 249 is an AAV9 VP1 capsid protein comprising VHH RGD peptide insertion and mutation N498I (amino acid numbering in reference to wild-type AAV9 capsid protein). In some aspects, the modified VP1 capsid protein set forth in SEQ ID NO: 249 is encoded by a nucleic acid sequence at least 80%, at least 85%, at least 90%, at least 95%, at least 96%, at least 97%, at least 98%, or at least 99%, or at least 100% (or any percentage in between) identical to SEQ ID NO: 274.

In some aspects, a modified VP2/VP3 capsid protein sequence is at least 80%, at least 85%, at least 90%, at least 95%, at least 96%, at least 97%, at least 98%, or at least 99%, or at least 100% (or any percentage in between) identical to SEQ ID NO: 262. In some aspects, SEQ ID NO: 262 is a modified AAV9 VP2/3 capsid protein comprising an RGD peptide insertion and mutation N498L (amino acid numbering in reference to wild-type AAV9 capsid protein). In some aspects, the VP2/VP3 capsid protein set forth in SEQ ID NO: 262 is encoded by a nucleic acid sequence at least 80%, at least 85%, at least 90%, at least 95%, at least 96%, at least 97%, at least 98%, or at least 99%, or at least 100% (or any percentage in between) identical to SEQ ID NO: 287.

In some aspects, a modified VP1 capsid protein sequence is at least 80%, at least 85%, at least 90%, at least 95%, at least 96%, at least 97%, at least 98%, or at least 99%, or at least 100% (or any percentage in between) identical to SEQ ID NO: 255. In some aspects, SEQ ID NO: 255 is an AAV9 VP1 capsid protein comprising VHH RGD peptide insertion. In some aspects, the modified VP1 capsid protein set forth in SEQ ID NO: 255 is encoded by a nucleic acid sequence at least 80%, at least 85%, at least 90%, at least 95%, at least 96%, at least 97%, at least 98%, or at least 99%, or at least 100% (or any percentage in between) identical to SEQ ID NO: 280.

In some aspects, a modified VP2/VP3 capsid protein sequence is at least 80%, at least 85%, at least 90%, at least 95%, at least 96%, at least 97%, at least 98%, or at least 99%, or at least 100% (or any percentage in between) identical to SEQ ID NO: 267. In some aspects, SEQ ID NO: 267 is a modified AAV9 VP2/3 capsid protein comprising RGD peptide insertion. In some aspects, the VP2/VP3 capsid protein set forth in SEQ ID NO: 267 is encoded by a nucleic acid sequence at least 80%, at least 85%, at least 90%, at least 95%, at least 96%, at least 97%, at least 98%, or at least 99%, or at least 100% (or any percentage in between) identical to SEQ ID NO: 292.

### Modified AAV Capsids

Modified AAV capsid proteins of the disclosure can be used to form AAV capsids, thereby forming a modified AAV capsid. The modified AAV capsid proteins provided herein may be used to construct modified, chimeric, and/or hybrid AAV capsids. An AAV capsid provided herein may comprise any combination of VP1, VP2, and VP3 sequences or any combination ov VP1 and VP2/VP3 proteins.

In some embodiments, a modified AAV capsid provided herein comprises a wild-type or modified VP1 capsid sequence at least 80%, at least 85%, at least 90%, at least 95%, at least 96%, at least 97%, at least 98%, or at least 99%, or at least 100% (or any percentage in between) identical to the VP1 sequences set forth in Table 1 and the SEQ ID NOs referenced therein. In some embodiments, a modified AAV capsid provided herein comprises a wild-type or modified VP2/VP3 capsid sequence at least 80%, at least 85%, at least 90%, at least 95%, at least 96%, at least 97%, at least 98%, or at least 99%, or at least 100% (or any percentage in between) identical to the VP2/VP3 sequences set forth in Table 1 and the SEQ ID NOs referenced therein. In some embodiments, a modified AAV capsid of the disclosure comprises a VP1 capsid protein selected from a sequence listed in Table 1 and a VP2/VP3 capsid protein selected from a sequence listed in Table 1.

In some aspects, a modified AAV capsid comprises a VP1 capsid protein sequence at least 80%, at least 85%, at least 90%, at least 95%, at least 96%, at least 97%, at least 98%, or at least 99%, or at least 100% (or any percentage in between) identical to SEQ ID NO: 53 and a VP2/VP3 capsid protein sequence at least 80%, at least 85%, at least 90%, at least 95%, at least 96%, at least 97%, at least 98%, or at least 99%, or at least 100% (or any percentage in between) identical to SEQ ID NO: 102. In some aspects, an AAV capsid having said sequences is referred to as LBV55. LBV55 comprises an AAV9 VP1 capsid protein comprising a VHH RGD peptide insertion and an AAV9 VP2/VP3 capsid protein.

In some aspects, a modified AAV capsid comprises a VP1 capsid protein sequence at least 80%, at least 85%, at least 90%, at least 95%, at least 96%, at least 97%, at least 98%, or at least 99%, or at least 100% (or any percentage in between) identical to SEQ ID NO: 75 and a VP2/VP3 capsid protein sequence at least 80%, at least 85%, at least 90%, at least 95%, at least 96%, at least 97%, at least 98%, or at least 99%, or at least 100% (or any percentage in between) identical to SEQ ID NO: 124. In some aspects, an AAV capsid having said sequences is referred to as LBV93. LBV93 comprises an Rh74 VP1 capsid protein comprising an mColQ peptide insertion and mutations F503I, G507R, and Y707C (amino acid numbering in reference to wild-type Rh74 capsid protein) and an Rh74 VP2/VP3 capsid protein comprising an RGD peptide insertion and mutations F503I, G507R, and Y707C (amino acid numbering in reference to wild-type Rh74 capsid protein).

In some aspects, a modified AAV capsid comprises a VP1 capsid protein sequence at least 80%, at least 85%, at least 90%, at least 95%, at least 96%, at least 97%, at least 98%, or at least 99%, or at least 100% (or any percentage in between) identical to SEQ ID NO: 245 and a VP2/VP3 capsid protein sequence at least 80%, at least 85%, at least 90%, at least 95%, at least 96%, at least 97%, at least 98%, or at least 99%, or at least 100% (or any percentage in between) identical to SEQ ID NO: 258. In some aspects, an AAV capsid having said sequences is referred to as LBV110. LBV110 comprises an Rh74 VP1 capsid protein comprising VHH RGD peptide insertion and mutations F503I, G507R, and Y707C (amino acid numbering in reference to wild-type Rh74 capsid protein) and an Rh74 VP2/VP3 capsid protein comprising an RGD peptide insertion and mutations F503I, G507R, and Y707C (amino acid numbering in reference to wild-type Rh74 capsid protein).

In some aspects, a modified AAV capsid comprises a VP1 capsid protein sequence at least 80%, at least 85%, at least 90%, at least 95%, at least 96%, at least 97%, at least 98%, or at least 99%, or at least 100% (or any percentage in between) identical to SEQ ID NO: 249 and a VP2/VP3 capsid protein sequence at least 80%, at least 85%, at least 90%, at least 95%, at least 96%, at least 97%, at least 98%, or at least 99%, or at least 100% (or any percentage in between) identical to SEQ ID NO: 262. In some aspects, an AAV capsid having said sequences is referred to as LBV114. LBV114 comprises an AAV9 VP1 capsid protein comprising a VHH RGD peptide insertion and mutation N498I (amino acid numbering in reference to wild-type AAV9 capsid protein) and an AAV9 VP2/VP3 capsid protein comprising an RGD peptide insertion and mutation N498L (amino acid numbering in reference to wild-type AAV9 capsid protein).

In some aspects, a modified AAV capsid comprises a VP1 capsid protein sequence at least 80%, at least 85%, at least 90%, at least 95%, at least 96%, at least 97%, at least 98%, or at least 99%, or at least 100% (or any percentage in between) identical to SEQ ID NO: 255 and a VP2/VP3 capsid protein sequence at least 80%, at least 85%, at least 90%, at least 95%, at least 96%, at least 97%, at least 98%, or at least 99%, or at least 100% (or any percentage in between) identical to SEQ ID NO: 267. In some aspects, an AAV capsid having said sequences is referred to as LBV121. LBV121 comprises an AAV9 VP1 capsid protein comprising a VHH RGD peptide insertion and an AAV9 VP2/VP3 capsid protein comprising an RGD peptide insertion.

A modified AAV capsid of the disclosure show improved transduction efficiency compared to the parental or wild-type AAV capsid. In some aspects, the improved transduction is in muscle tissue. In some aspects, the muscle tissue is skeletal muscle, smooth muscle, or cardiac muscle. In some embodiments, the transduction efficiency of a modified muscle-targeted AAV capsid provided herein in a muscle cell is increased by at least about 10%, at least about 20%, at least about 30%, at least about 40%, at least about 50%, at least about 60%, at least about 70%, at least about 80%, or at least about 90% compared to the parental AAV capsid as determined by immunofluorescence. In some embodiments, the transduction efficiency of a modified muscle-targeted AAV capsid provided herein in a muscle cell is increased by at least about 1.1-fold, at least about 1.2-fold, at least about 1.3-fold, at least about 1.4-fold, at least about 1.5-fold, at least about 2-fold, at least about 5-fold, at least about 10-fold, at least about 20-fold, at least about 30-fold, at least about 40-fold, or at least about 50-fold compared to the parental AAV capsid as determined by immunofluorescence.

### Viral Vectors and Pharmaceutical Compositions

The AAV capsids of the disclosure may be used in any suitable AAV viral vector. The AAV capsid surrounds a small, single-stranded DNA genome of approximately 4.8 kilobases (kb). The genome of an AAV contains three genes, Rep (Replication), Cap (Capsid), and aap (Assembly) flanked by inverted terminal repeats (ITRs) that are required for genome replication and packaging. See Naso et al., BioDrugs. 2017; 31(4): 317-334. Recombinant AAV vectors lack the viral gene and instead comprise a transgene flanked by the two viral ITRs.

Thus, in another aspect, provided herein is an AAV viral vectors comprising a modified AAV capsid provided herein. In some embodiments, the AAV viral vector comprises a transgene.

Also provided herein is a pharmaceutical composition comprising an AAV viral vector comprising a modified AAV capsid provided herein and a pharmaceutically acceptable carrier. A pharmaceutical composition may be formulated for any suitable route of administration, including, for example, intraveneous, intrathecal, intracranial, or intraocular administration. Pharmaceutical compositions for use as disclosed herein may comprise a protein(s) or a polynucleotide encoding the protein(s), optionally comprised in an AAV, which is optionally also immune orthogonal, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the disclosure may be formulated for routes of administration, such as e.g., oral, enteral, topical, transdermal, intranasal, and/or inhalation; and for routes of administration via injection or infusion such as, e.g., intravenous, intramuscular, subpial, intrathecal, intraparenchymal, intrathecal, intrastriatal, subcutaneous, intradermal, intraperitoneal, intratumoral, intravenous, intraocular, and/or parenteral administration. In certain embodiments, the compositions of the present disclosure are formulated for intracerebral or intrastriatal administration.

### Methods of Use

The disclosure provides a method of delivering a transgene or NOI to a tissue of interest in a subject comprising administering an AAV viral vector comprising a modified AAV capsid protein of the disclosure. In one embodiment, the disclosure provides a method of modifying an activity of a protein encoded by an RNA molecule comprising contacting an AAV viral vector of the disclosure and the RNA molecule under conditions suitable for binding of one or more of the guide RNA or the RNA-binding protein or the fusion protein (or a portion thereof) to the RNA molecule.

The disclosure provides a method of modifying the level of expression of an RNA molecule of the disclosure or a protein encoded by the RNA molecule comprising contacting an AAV viral vector of the disclosure and a cell comprising the RNA molecule under conditions suitable for binding of one or more of the guide RNA or the RNA-binding protein or fusion protein (or a portion thereof) to the RNA molecule. In some embodiments, the cell is in vivo, in vitro, ex vivo or in situ. In some embodiments, the AAV viral vector of the disclosure comprises a guide RNA of the disclosure and an RNA-binding protein or fusion protein of the disclosure.

The disclosure provides a method of modifying an activity of a protein encoded by an RNA molecule comprising contacting an AAV viral vector of the disclosure and a cell comprising the RNA molecule under conditions suitable for binding of one or more of the guide RNA or the RNA-binding protein or fusion protein (or a portion thereof) to the RNA molecule.

The disclosure provides a method of modifying the level of expression of an RNA molecule of the disclosure or a protein encoded by the RNA molecule comprising contacting an AAV viral vector of the disclosure and the RNA molecule under conditions suitable for RNA nuclease activity wherein the RNA-binding protein or fusion protein induces a break in the RNA molecule.

The disclosure provides a method of modifying an activity of a protein encoded by an RNA molecule comprising contacting an AAV viral vector of the disclosure and the RNA molecule under conditions suitable for RNA nuclease activity wherein the RNA-binding protein or fusion protein induces a break in the RNA molecule.

The disclosure provides a method of modifying a level of expression of an RNA molecule of the disclosure or a protein encoded by the RNA molecule comprising contacting an AAV viral vector of the disclosure and a cell comprising the RNA molecule under conditions suitable for RNA nuclease activity wherein the RNA-binding protein or fusion protein induces a break in the RNA molecule. In some embodiments, the cell is in vivo, in vitro, ex vivo or in situ. In some embodiments, the AAV viral vector of the disclosure comprises a guide RNA of the disclosure and an RNA-binding fusion protein of the disclosure.

The disclosure provides a method of modifying an activity of a protein encoded by an RNA molecule comprising contacting an AAV viral vector of the disclosure and a cell comprising the RNA molecule under conditions suitable for RNA nuclease activity wherein the RNA-binding protein or fusion protein induces a break in the RNA molecule. In some embodiments, the cell is in vivo, in vitro, ex vivo or in situ. In some embodiments, an AAV viral vector of the disclosure comprises a guide RNA or a single guide RNA of the disclosure and a nucleic acid sequence encoding an RNA-binding protein or fusion protein of the disclosure.

The disclosure provides a method of treating a subject having a disease or disorder comprising administering to a subject a therapeutically effective amount of an AAV viral vector or a pharmaceutical composition of the disclosure. In some aspects, the disease or disorder muscular and/or neuromuscular disease or disorder. In some aspects, the muscular and/or neuromuscular disorder is muscular dystrophy or myotonic dystrophy.

The disclosure provides a method of treating a disease in a patient in need of such treatment comprising administering to the patient a therapeutically effective amount of an AAV viral vector or a pharmaceutical composition of the disclosure, wherein an AAV viral vector or a pharmaceutical composition comprises a vector comprising a guide RNA of the disclosure and a nucleic acid sequence encoding an RNA-binding protein or an RNA-binding protein fusion protein of the disclosure, wherein an AAV viral vector or a pharmaceutical composition modifies, reduces, destroys, knocks down or ablates a level of expression of a toxic repeat RNA (compared to the level of expression of a toxic repeat RNA treated with a non-targeting (NT) control or compared to no treatment). In another embodiment, the level of reduction is 1-fold or greater. In another embodiment, the level of reduction is 2-fold, 3-fold, 4-fold, 5-fold, 6-fold, 7-fold, 8-fold, 9-fold or 10-fold. In another embodiment, the level of reduction is 10-fold or greater. In another embodiment, the level of reduction is between 10-fold and 20-fold. In another embodiment, the level of reduction is 11-fold, 12-fold, 13-fold, 14-fold, 15-fold, 16-fold, 17-fold, 18-fold, 19-fold, or 20-fold. In another embodiment, the gene therapy compositions disclosed herein when administered to a patient lead to 20%-100% destruction of the toxic repeat RNA. In one embodiment, the % elimination of the toxic repeat RNA is any of 20-99%, 25%-99%, 50%-99%, 80%-99%, 90%-99%, 95%-99%. In one embodiment, the % elimination is 90%, 91%, 92%, 93%, 94%, 95%, 96%, 97%, 98%, or 99%. In another embodiment, % elimination is complete elimination or 100% elimination of the toxic repeat RNA.

In some embodiments of the methods of the disclosure, a subject of the disclosure has been diagnosed with a disease to be treated. In some embodiments, the subject of the disclosure presents at least one sign or symptom of a disorder or disease to be treated. In some embodiments, the subject of the disclosure presents at least one sign or symptom of a disease.

In some embodiments of the methods of the disclosure, a subject of the disclosure is female. In some embodiments of the methods of the disclosure, a subject of the disclosure is male. In some embodiments, a subject of the disclosure has two XX or XY chromosomes. In some embodiments, a subject of the disclosure has two XX or XY chromosomes and a third chromosome, either an X or a Y.

In some embodiments of the methods of the disclosure, a subject of the disclosure is a neonate, an infant, a child, an adult, a senior adult, or an elderly adult. In some embodiments of the methods of the disclosure, a subject of the disclosure is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 days old. In some embodiments of the methods of the disclosure, a subject of the disclosure is at least 1, 2, 3, 4, 5, 6, 7, 8, 9, 10, 11 or 12 months old. In some embodiments of the methods of the disclosure, a subject of the disclosure is at least 1, 2, 3, 4, 5, 6, 7, 8, 9, 10, 15, 20, 25, 30, 35, 40, 45, 50, 55, 60, 65, 70, 75, 80, 85, 90, 95, 100 or any number of years or partial years in between of age.

In some embodiments of the methods of the disclosure, a subject of the disclosure is a mammal. In some embodiments, a subject of the disclosure is a non-human mammal.

In some embodiments of the methods of the disclosure, a subject of the disclosure is a human.

In some embodiments of the methods of the disclosure, a therapeutically effective amount comprises a single dose of a composition of the disclosure. In some embodiments, a therapeutically effective amount comprises a therapeutically effective amount comprises at least one dose of a composition of the disclosure. In some embodiments, a therapeutically effective amount comprises a therapeutically effective amount comprises one or more dose(s) of a composition of the disclosure.

In some embodiments of the methods of the disclosure, a therapeutically effective amount eliminates a sign or symptom of the disease or disorder. In some embodiments, a therapeutically effective amount reduces a severity of a sign or symptom of the disease or disorder.

In some embodiments of the methods of the disclosure, a therapeutically effective amount eliminates the disease or disorder.

In some embodiments of the methods of the disclosure, a therapeutically effective amount prevents an onset of a disease or disorder. In some embodiments, a therapeutically effective amount delays the onset of a disease or disorder. In some embodiments, a therapeutically effective amount reduces the severity of a sign or symptom of the disease or disorder. In some embodiments, a therapeutically effective amount improves a prognosis for the subject.

In some embodiments of the methods of the disclosure, a composition of the disclosure is administered to the subject via intracerebral administration. In some embodiments, the composition of the disclosure is administered to the subject by an intrastriatal route. In some embodiments, the composition of the disclosure is administered to the subject by a stereotaxic injection or an infusion. In some embodiments, the composition is administered to the brain. In some embodiments of the methods of the disclosure, a composition of the disclosure is administered to the subject locally.

In some embodiments, the compositions disclosed herein are formulated as pharmaceutical compositions. Briefly, pharmaceutical compositions for use as disclosed herein may comprise a protein(s) or a polynucleotide encoding the protein(s), optionally comprised in an AAV, which is optionally also immune orthogonal, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the disclosure may be formulated for routes of administration, such as e.g., oral, enteral, topical, transdermal, intranasal, and/or inhalation; and for routes of administration via injection or infusion such as, e.g., intravenously, intrathecally, intracerebrally, intraventricularly, intranasally, intratracheally, intra-aurally, intra-ocularly, or peri-ocularly, orally, rectally, transmucosally, inhalationally, transdermally, parenterally, subcutaneously, intradermally, intramuscularly, intracisternally, intranervally, intrapleurally, topically, intralymphatically, intracisternally; such introduction may also be intra-arterial, intracardiac, subventricular, epidural, intracerebral, intracerebroventricular, subretinal, intravitreal, intraarticular, intraperitoneal, intrauterine, systemically or any combination thereof.

In some aspects, enhanced transduction in muscle tissue occurs following delivery of AAV viral vectors comprising modified AAV capsid proteins. In some aspects, enhanced transduction in ocular tissue occurs following subretinal delivery of AAV vectors comprising modified AAV capsid proteins relative to AAV capsids comprising unmodified, wild-type, or parental AAV capsid proteins. In some aspects, enhanced transduction in neural tissue occurs following delivery of AAV vectors comprising modified AAV capsid proteins relative to AAV capsids comprising unmodified, wild-type, or parental AAV capsid proteins. In some aspects, reduced or no liver or hepatocyte transduction occurs following systemic delivery of AAV vectors comprising modified AAV capsids relative to AAV capsids comprising unmodified, wild-type, or parental AAV capsid proteins. In some aspects, reduced or no neutralizing antibody binding occurs following systemic delivery of AAV vectors comprising modified AAV capsids relative to AAV capsids comprising unmodified, wild-type, or parental AAV capsid proteins. In some aspects, neutralizing antibody titer is minimal following delivery of AAV vectors comprising modified AAV capsids relative to AAV capsids comprising unmodified, wild-type, or parental AAV capsid proteins. In some aspects, neutralizing antibody titer is reduced compared to delivery of AAV vectors comprising nonmodified AAV capsids relative to AAV capsids comprising unmodified, wild-type, or parental AAV capsid proteins.

### Cells

In some embodiments of the compositions and methods of the disclosure, a cell of the disclosure is a prokaryotic cell.

In some embodiments of the compositions and methods of the disclosure, a cell of the disclosure is a eukaryotic cell. In some embodiments, the cell is a mammalian cell. In some embodiments, the cell is a bovine, murine, feline, equine, porcine, canine, simian, or human cell. In some embodiments, the cell is a non-human mammalian cell such as a non-human primate cell.

In some embodiments, a cell of the disclosure is a somatic cell. In some embodiments, a cell of the disclosure is a germline cell. In some embodiments, a germline cell of the disclosure is not a human cell.

In some embodiments of the compositions and methods of the disclosure, a cell of the disclosure is a stem cell. In some embodiments, a cell of the disclosure is an embryonic stem cell. In some embodiments, an embryonic stem cell of the disclosure is not a human cell. In some embodiments, a cell of the disclosure is a multipotent stem cell or a pluripotent stem cell. In some embodiments, a cell of the disclosure is an adult stem cell. In some embodiments, a cell of the disclosure is an induced pluripotent stem cell (iPSC). In some embodiments, a cell of the disclosure is a hematopoietic stem cell (HSC).

In some embodiments of the compositions and methods of the disclosure, a somatic cell of the disclosure is a muscle cell. In some embodiments, a muscle cell of the disclosure is a myoblast or a myocyte. In some embodiments, a muscle cell of the disclosure is a cardiac muscle cell, skeletal muscle cell or smooth muscle cell. In some embodiments, a muscle cell of the disclosure is a striated cell. In one embodiment, a cell or cells of a patient treated with compositions disclosed herein include, without limitation, skeletal muscle (developing and mature muscle fibers and satellite cells), neuromuscular junction, cardiomyocytes, smooth muscle cells, peripheral nervous system (neurons), peripheral motor neurons, and/or sensory neurons.

In some embodiments of the compositions and methods of the disclosure, a somatic cell of the disclosure is a fibroblast or an epithelial cell. In some embodiments, an epithelial cell of the disclosure forms a squamous cell epithelium, a cuboidal cell epithelium, a columnar cell epithelium, a stratified cell epithelium, a pseudostratified columnar cell epithelium or a transitional cell epithelium. In some embodiments, an epithelial cell of the disclosure forms a gland including, but not limited to, a pineal gland, a thymus gland, a pituitary gland, a thyroid gland, an adrenal gland, an apocrine gland, a holocrine gland, a merocrine gland, a serous gland, a mucous gland and a sebaceous gland. In some embodiments, an epithelial cell of the disclosure contacts an outer surface of an organ including, but not limited to, a lung, a spleen, a stomach, a pancreas, a bladder, an intestine, a kidney, a gallbladder, a liver, a larynx or a pharynx. In some embodiments, an epithelial cell of the disclosure contacts an outer surface of a blood vessel or a vein.

In some embodiments of the compositions and methods of the disclosure, a somatic cell of the disclosure is a primary cell.

In some embodiments of the compositions and methods of the disclosure, a somatic cell of the disclosure is a cultured cell.

In some embodiments of the compositions and methods of the disclosure, a somatic cell of the disclosure is in vivo, in vitro, ex vivo or in situ.

In some embodiments of the compositions and methods of the disclosure, a somatic cell of the disclosure is autologous or allogeneic.

### EXAMPLES

The examples provided in this section are intended for illustration only and are not intended to limit the invention.

### Example 1: VP1-Specific Display of Targeting Peptides

The use of a targeting peptide can increase transfection efficacy in a certain tissue or cell type. However, how a peptide is displayed on an AAV capsid influences the effect it has.

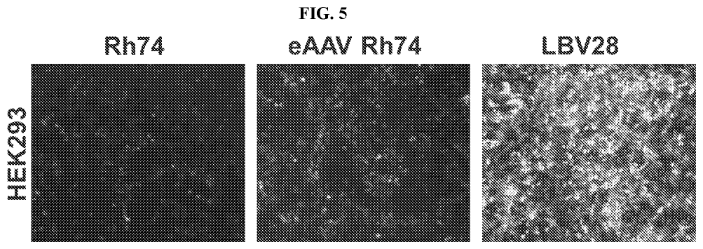

FIG. 5 show immunofluorescence images of HEK293 cells transfected with an AAV Rh74 particle comprising a peptide insertion. Both eAAV Rh74 and LBV28 contain the insulin receptor targeting peptide, S519. In eAAV Rh74 the insertion site VR8 of VP1, VP2, and VP3 at a 1 to 10 of mutant to wild-type protein. LBV28 has the peptide inserted specifically in VR8 VP1 and not in VP2 or VP3. These data show that VP1-specific insertion of LBV28 results in greater effectiveness of the targeting peptide. Without wishing to be bound by theory, it is hypothesized that the VP1-specific insertion allows the peptide to be displayed more freely, which results in greater effectiveness, while on the other hand problems with capsid assembly limit the use of linkers when a peptide is inserted into all the VP1-3 proteins.

### Example 2: Generation and Testing of Novel Muscle-Targeted AAV Capsids

Novel and improved muscle-targeting capsid proteins from AAV9, AAVRh74, and AAVpo1. were rationally designed. Rationally targeted capsid mutations and insertions were cloned and HEK293 cells and iodixanol gradient ultra-centrifuge purification were used to produce GFP and Luciferase reporter viruses. Coomassie stain was used to verify that the preparations were free of protein contaminants. Endotoxin levels must be less than 1.5 EU/ml. Titers were measured by ITR qPCR and engineered capsid must produce at 25% of parental capsid to progress to in vitro studies. C2C12 myoblast cells were used to evaluate potential muscle-targeting of engineered capsids. Cells were cultured Dulbecco's Modified Eagle Medium (DMEM) with 10% FBS and transduction was visually assessed by GFP signal (both the percentage of GFP+ cells and level of GFP expression) using a fluorescent microscope. Briefly, C2C12 cells were seeded at density of 5E4 cells per well in 48 well plate on Day 1. On Day 3, cells were transduced with AAVs at multiplicity of infection ((MOI); number of viral genomes delivered divided by total number of cells) of 1E5 and 1E6 based on 1E5 cells per well. GFP signal was monitored on days 4-10.

AAV capsids having modified AAV capsid proteins comprising RGD peptide insertions were evaluated. LBV54 comprises a flexible linker RGD peptide insertion in AAV9 VR VIII (VR8). LBV55 comprises a VHH RGD peptide insertion in AAV9 VR VIII (VR8). LBV56 comprises a GP2 RGD peptide insertion in AAV9 VR VIII (VR8). LBV57 comprises a cyclic peptide RGD peptide insertion in AAV9 VR VIII (VR8). LBV58 comprises a knottin RGD peptide insertion in AAV9 VR VIII (VR8) (FIG. 8). AAV capsids having modified AAV capsid proteins comprising a ColQ peptide insertion was also evaluated (LBV31).

Vector genome yield and the peptides are summarized in 2.

TABLE 2
Alternative RGD Display Scaffolds

| | | Titer | Productivity |
|---|---|---|---|
| Name | Genome | (vg/mL) | (vg/cell) |
| AAV9 | tCAG Firefly Luciferase | 2.16E+14 | 1.89E+05 |
| | P2A Clover3 | | |
| LBV54 | tCAG Firefly Luciferase | 2.06E+14 | |
| | P2A Clover3 | | |
| LBV55 (AAV9- | tCAG Firefly Luciferase | 5.71E+13 | 5.00E+04 |
| based) | P2A Clover3 | | |
| AAVpo1 | tCAG Firefly Luciferase | 1.67E+14 | 1.46E+05 |
| | P2A Clover3 | | |
| LBV56 | tCAG Firefly Luciferase | 7E+11 | |
| | P2A Clover3 | | |
| LBV57 | tCAG Firefly Luciferase | 4.69E+11 | |
| | P2A Clover3 | | |
| LBV58 | tCAG Firefly Luciferase | 1.18E+12 | |
| | P2A Clover3 | | |
| AAV2 | tCAG Firefly Luciferase | 1.24E+11 | |
| | P2A Clover3 | | |
| LBV31 (AAVpo1- | tCAG Firefly Luciferase | 1.24E+14 | 1.09E+05 |
| based) | P2A Clover3 | | |
| AAVRh74 | CMV GFP | 1.23E+14 | 1.08E+05 |
| LBV30 (AAVRh74- | CMV GFP | 8.10E+13 | 7.09E+04 |
| based) | | | |

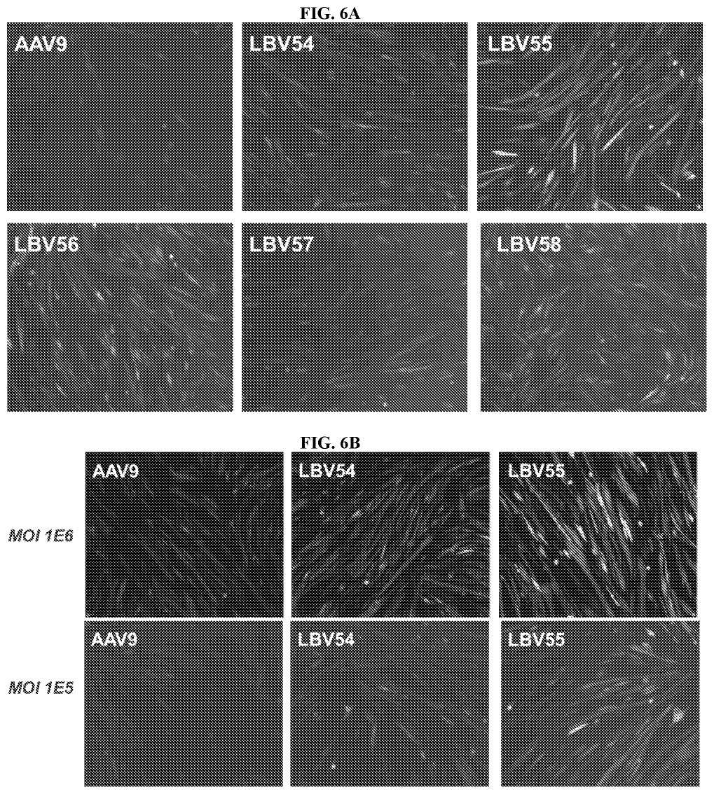

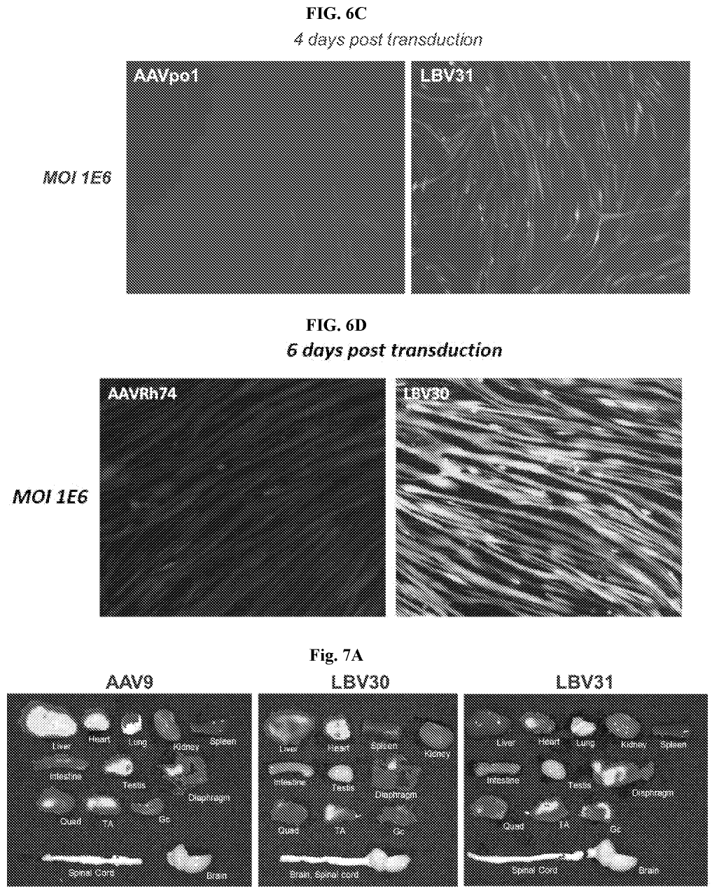

101941 Results are shown in FIGS. 6A through 6D). A significant enhancement of C2C12 transduction was observed 5 days post-transduction for VHHT RGD insertion capsid LBV55 compared to wild-type AAV9 (FIG. 6A and FIG. 6B3). Further, a significant transduction enhancement was observed 4 days post-transduction for modified PO1 capsid LBV31 relative to wild-type AAVPO1 (FIG. 6C). An enhancement in transduction for Rh774-derived LBV30 was observed 6 days post transduction relative to Rh74.

### Example 3: Ex Vivo Imaging of AAV9, LBV30, and LBV31

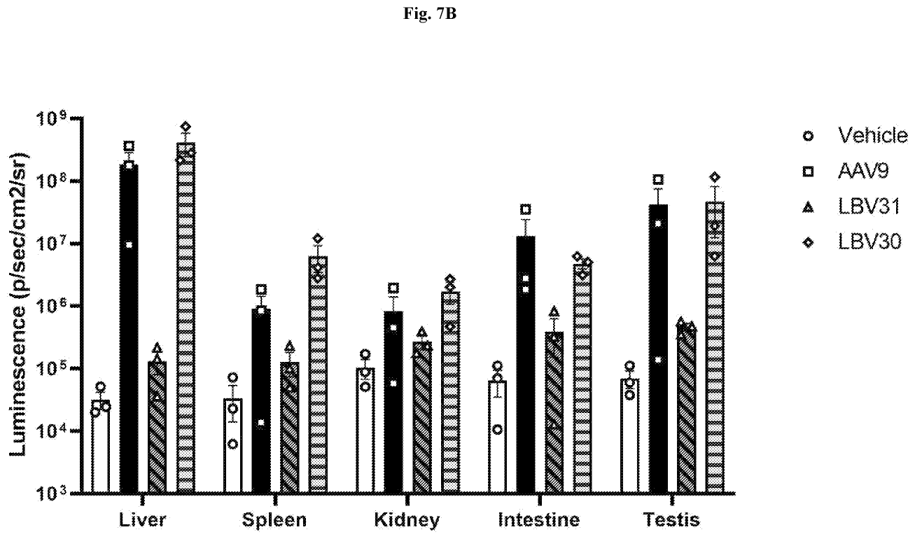

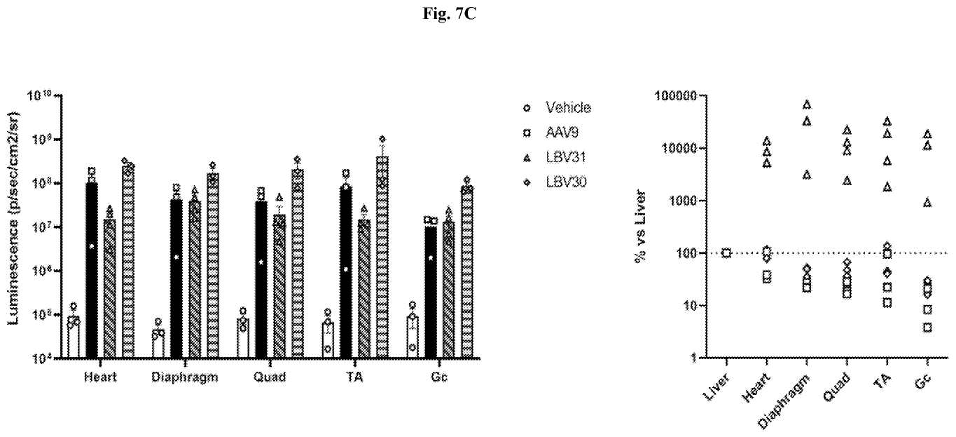

In order to evaluate in vivo targeting of the modified AAV capsids, mice were injected intravenously with reporter viruses at a dose of 1E12 vg/mouse. Ex vivo imaging of skeletal, smooth, cardiac muscles, CNS, liver, and other internal organs was carried out 4 weeks post injection. Results are shown in FIG. 7A-7C. FIG. 7A shows representative images comparing the modified capsid to the LBV30 and LBV31 variants. LBV30 is an AAV Rh74 capsid comprising a modified VP1 capsid protein having a ColQ peptide insertion and a VP2/VP3 capsid protein having an RGD peptide insertion. LBV31 is an AAV PO1 capsid comprising a modified VP1 capsid protein having a ColQ peptide insertion and a VP2/VP3 capsid protein having an RGD peptide insertion. Data in FIG. 7B indicates that LBV31 has limited transduction of off-target tissues, while data in FIG. 7C indicates that LBV31 transduces target muscles. These data show that LBV31 shows a favorable profile of reasonable skeletal, smooth, and cardiac muscle transduction and little to no transduction of multiple non-muscle tissues (most importantly no transduction of the liver). LBV30 shows increased transduction of some muscles relative to AAV9. However, there is also a significant amount of liver transduction.

### Example 4: Liver De-Targeting Mutations in Modified AAV Capsid Proteins

LBV55 displayed enhanced muscle transduction. To enhance liver de-targeting, mutations F501I, G505R, and Y706C were introduced into VP1 and VP2/VP3 of LBV55 to generate LBV91. Additionally, LBV92 was generated from LBV30 by incorporating mutations F501I, G505R, and Y706C into VP1 and VP2/VP3. Viral Vectors were prepared (Table 3) comprising luciferase reporting vector pAAV-tCAG Firefly Luciferase-P2A-Clover3 WPRE and administered to mice (Table 4). Mouse IV injection of Luciferase reporter viruses, 1E12 vg/mouse. Ex vivo imaging of skeletal, smooth, cardiac muscles, CNS, liver, and other internal organs at 2-4 weeks post injection.

TABLE 3
AAV Vectors used in Study

| | VP1 | VP2/VP3 | |
|---|---|---|---|
| AAV9 | AAV9 | AAV9 | |
| LBV55 | AAV9 VR8 VHH | AAV9 | Muscle targeting |
| | RGD | | |
| LBV91 | AAV9 F501I | AAV9 F501I | Muscle targeting + |
| | G505R VR8 VHH | G505R Y706C | liver de-targeting |
| | RGD Y706C | | |
| Rh74 | Rh74 | Rh74 | |
| LBV92 | Rh74 F301I | Rh74 F503I | Muscle targeting + |
| | G507R VR8 ColQ | G507R Vr8 RGD | liver de-targeting |
| | Y706C | (P1) Y708C | |

TABLE 4
Study criteria including time, dose, and n

| | Test | | | Dose | Timepoint | n |
|---|---|---|---|---|---|---|
| Group | substance | Serotype | Strain | (vg) | (wks) | 4 |
| 1 | VP00148 | AAV9 | FVB | 1E12 | 2 | 4 |
| 2 | VP00074 | LBV55 | FVB | 1E12 | 2 | 4 |
| 3 | VP00218 | LBV91 | FVB | 1E12 | 2 | 4 |
| 4 | VP00217 | Rh74 | FVB | 1E12 | 2 | 4 |
| 5 | VP00219 | LBV92 | FVB | 1E12 | 2 | 4 |
| 6 | Vehicle | | FVB | n/a | 2 | 3 |

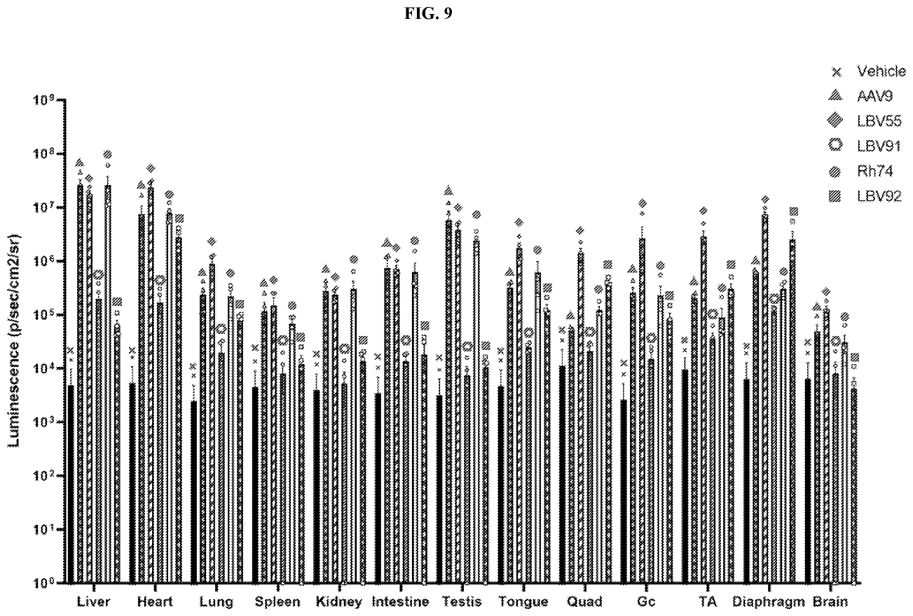

LBV91 showed near complete liver de-targeting, but muscle transduction was also reduced relative to LBV55. LBV92 showed maintained or enhanced muscle transduction and liver de-targeting (less than 1% of Rh74 or AAV9). FIG. 9. LBV92 therefore achieves: 1) Increases in muscle transduction (Quadricep (quad), tibialis anterior (TA), and diaphragm); 2) greater than 2 order of magnitude decrease in liver transduction; and 3) no increased transduction in other organs.

We claim:

1. A modified AAV capsid protein comprising a modified variable region (VR) VIII.

2. The modified AAV capsid protein of claim 1, wherein the capsid protein is a VP1, VP2, or VP3 capsid protein.

3. The modified AAV capsid protein of claim 1, wherein the modified VR VIII comprises a peptide insertion.

4. The modified AAV capsid protein of claim 3, wherein the peptide insertion comprises an RGD-motif peptide insertion.

5. The modified AAV capsid protein of claim 4, wherein the RGD-motif insertion comprises RGDLGLS (SEQ ID NO: 303), RGDLSTP (SEQ ID NO: 304), SNSRGDYNSL (SEQ ID NO: 305), ENRRGDFNNT (SEQ ID NO: 306), SRGDYNSL (SEQ ID NO: 307), RGDYNSL (SEQ ID NO: 308), RGDLST (SEQ ID NO: 309), or RGDYVGL (SEQ ID NO: 310).

6. The modified AAV capsid protein of claim 4, wherein the RGD-motif insertion comprises a variable domain of camelid heavy-chain-only antibody (VHH) RGD peptide.

7. The modified AAV capsid protein of claim 6, wherein the VHH RGD peptide comprises an amino acid sequence at least 85%, 90%, 95%, 96%, 97%, 98%, 99% or 100% identical to the amino acid sequence EVQLQASGGGFVQAGGSLRLSCAVSGRGDLSTPSYGMHWVRQAPGKEREFVAGISR GDYNSLYYADSVQGRFTISRDNAKNTVYLQMNSLKPEDTATYYCAENRRGDFNNT YWGQGTQVTVSS (SEQ ID NO: 316).

8. The modified AAV capsid protein of claim 3, wherein the peptide insertion comprises an acetylcholinesterase collagenic tail (ColQ) peptide.

9. The modified AAV capsid protein of claim 8, wherein the ColQ peptide comprises an amino acid sequence at least 85%, 90%, 95%, 96%, 97%, 98%, 99% or 100% identical to the amino acid sequence TPFYPVGYTVKQPGTCGDGVLQPGEECDDGNPDVSDGCIDCHRAYCGDGYRHQGV EDCDGSDFGYLTCETYLPGSYGDLRCTQYCSIDSTPCRYFT (SEQ ID NO: 302).

10. The modified AAV capsid protein of claim 3, wherein the peptide insertion comprises one or more linker sequences at the N-terminus or C-terminus of the inserted peptide.

11. The modified AAV capsid protein of claim 8, wherein the linker sequence comprises GGGGS (SEQ ID NO: 311), GGGGSGGGGS (SEQ ID NO: 312); GGGGSGGGGSGGGGS(SEQ ID NO: 313); GGGGSGGGGSGGGGSGGGGS (SEQ ID NO: 314); or GGGGSGGGGSGGGGSGGGGSGGGGS (SEQ ID NO: 315).

12. The modified AAV capsid protein of claim 1, wherein the AAV serotype is AAV9 or AAVRh74.

13. The modified AAV capsid protein of claim 1, further comprising one or more amino acid mutations.

14. The modified AAV capsid protein of claim 13, wherein the one or more amino acid mutations reduce liver transduction.

15. The modified AAV capsid protein of claim 13, wherein the mutations comprise:
i) at least one of F503I, G507I, Y707C, and/or Y708C of Rh74, or
ii) N498I of AAV9.

16. The modified AAV capsid protein of any one of the preceding claims, wherein the capsid protein comprises an amino acid sequence at least 85%, 90%, 95%, 96%, 97%, 98%, 99% or 100% identical to the amino acid sequence set forth in any one of SEQ ID NOs: 53, 75, 102, 124, 245, 249, 255, 258, 262, or 267.

17. A modified AAV capsid protein comprising an amino acid sequence at least 85%, 90%, 95%, 96%, 97%, 98%, 99% or 100% identical to the amino acid sequence set forth in any one of SEQ ID NOs: 53, 75, 102, 124, 245, 249, 255, 258, 262, or 267.

18. The modified AAV capsid protein of claim 17, wherein the capsid protein is encoded by a nucleic acid sequence at least 85%, 90%, 95%, 96%, 97%, 98%, 99% or 100% identical to the amino acid sequence set forth in any one of SEQ ID NOs: 151, 173, 199, 221, 270, 274, 280, 283, 287, or 292.

19. An AAV capsid comprising one or more AAV capsid proteins according to any one of claims 1-18.

20. The AAV capsid of claim 19, wherein the AAV capsid has enhanced transduction in a targeted tissue or cell type relative to other tissue or cell types.

21. The AAV capsid of claim 20, wherein the targeted tissue type is muscular tissue or muscle cells.

22. The AAV capsid of claim 20, wherein the AAV capsid has enhanced transduction in muscle tissue.

23. The AAV capsid of claim 19, wherein the AAV capsid has reduced transduction in non-targeted tissues or cell types.

24. The AAV capsid of claim 23, wherein the non-targeted tissues include liver, lung, kidney, brain, spleen, intestine, spinal cord, or reproductive organs.

25. The AAV capsid of claim 19, wherein the transduction in muscle tissue is enhanced by at least about 10%, about 20%, about 30%, about 40%, about 50%, about 100%, about 200%, or about 300% compared the parental and/or unmodified AAV capsid.

26. The AAV capsid of claim 19, wherein the capsid comprises:
i) a VP1 capsid protein comprising an amino acid sequence at least 85%, 90%, 95%, 96%, 97%, 98%, 99% or 100% identical to the amino acid sequence set forth in SEQ ID NO: 53; and
ii) a VP2/VP3 capsid protein comprising an amino acid sequence at least 85%, 90%, 95%, 96%, 97%, 98%, 99% or 100% identical to the amino acid sequence set forth in SEQ ID NO: 102.

27. The AAV capsid of claim 19, wherein the capsid comprises:
i) a VP1 capsid protein comprising an amino acid sequence at least 85%, 90%, 95%, 96%, 97%, 98%, 99% or 100% identical to the amino acid sequence set forth in SEQ ID NO: 75; and
ii) a VP2/VP3 capsid protein comprising an amino acid sequence at least 85%, 90%, 95%, 96%, 97%, 98%, 99% or 100% identical to the amino acid sequence set forth in SEQ ID NO: 124.

28. The AAV capsid of claim 19, wherein the capsid comprises:
i) a VP1 capsid protein comprising an amino acid sequence at least 85%, 90%, 95%, 96%, 97%, 98%, 99% or 100% identical to the amino acid sequence set forth in SEQ ID NO: 245; and
ii) a VP2/VP3 capsid protein comprising an amino acid sequence at least 85%, 90%, 95%, 96%, 97%, 98%, 99% or 100% identical to the amino acid sequence set forth in SEQ ID NO: 258.

29. The AAV capsid of claim 19, wherein the capsid comprises:
i) a VP1 capsid protein comprising an amino acid sequence at least 85%, 90%, 95%, 96%, 97%, 98%, 99% or 100% identical to the amino acid sequence set forth in SEQ ID NO: 249; and
ii) a VP2/VP3 capsid protein comprising an amino acid sequence at least 85%, 90%, 95%, 96%, 97%, 98%, 99% or 100% identical to the amino acid sequence set forth in SEQ ID NO: 262.

30. The AAV capsid of claim 19, wherein the capsid comprises:
i) a VP1 capsid protein comprising an amino acid sequence at least 85%, 90%, 95%, 96%, 97%, 98%, 99% or 100% identical to the amino acid sequence set forth in SEQ ID NO: 255; and
ii) a VP2/VP3 capsid protein comprising an amino acid sequence at least 85%, 90%, 95%, 96%, 97%, 98%, 99% or 100% identical to the amino acid sequence set forth in SEQ ID NO: 267.

31. A vector comprising a nucleic acid sequence encoding a modified AAV capsid protein of claim 1.

32. The vector of claim 31, wherein the nucleic acid sequence encoding a modified AAV capsid protein is operably linked to regulatory element that controls expression of the capsid protein in a host cell.

33. An AAV viral vector comprising the modified capsid protein of any one of claims 1-18 or the AAV capsid of claim 18.

34. The AAV viral vector of claim 33, wherein the AAV viral vector comprises a recombinant AAV (rAAV) vector encoding a therapeutic transgene or nucleotide sequence of interest (NOI).

35. A cell comprising the vector of claim 31 or the AAV viral vector of claim 33.

36. A pharmaceutical composition comprising the AAV viral vector of claim 33 and at least one pharmaceutically acceptable excipient and/or additive.

37. A method of providing a therapeutic transgene or protein to a subject, comprising administering to the subject the AAV viral vector of claim 33 or the pharmaceutical composition of claim 36.

38. A method of treating a subject having a disease and/or disorder, the method comprising administering to the subject at least one therapeutically effective amount of the AAV viral vector of claim 33 or the pharmaceutical composition of claim 36.

39. The method of claim 38, wherein the disease and/or disorder is a muscular and/or neuromuscular disorder.

40. The method of claim 39, wherein the muscular and/or neuromuscular disorder is muscular dystrophy or myotonic dystrophy.

41. The method of any one of claims 37-40, wherein the AAV viral vector or the pharmaceutical composition is administered to the subject intravenously, intrathecally, intracerebrally, intraventricularly, intranasally, intratracheally, intra-aurally, intra-ocularly, or peri-ocularly, orally, rectally, transmucosally, inhalationally, transdermally, parenterally, subcutaneously, intradermally, intramuscularly, intracisternally, intranervally, intrapleurally, topically, intralymphatically, intracisternally or intranerve.
